SEQUENCE LISTING (2 sequences)

SEQ ID NO: 1 - DNA, 915 bp, synthetic construct
  source 1..915: mol_type = other DNA; note = NKp46
atgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtccaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttga

SEQ ID NO: 2 - protein, 304 aa, synthetic construct
  source 1..304: note = NKp46
MSSTLPALLCVGLCLSQRISAQQQTLPKPFIWAEPHFMVPKEKQVTICCQGNYGAVEYQLHFEGSLFAVDRPKPPERINKVQFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

(19) United States
(12) Patent Application Publication
(10) Pub. No.: US 2026/0315534 A1
(43) Pub. Date: Oct. 15, 2026

(54) METHOD FOR EXPANDING CD56+/CD3- CELLS

(71) Applicant: TAKEDA PHARMACEUTICAL COMPANY LIMITED, Osaka (JP)

(72) Inventors: Yoshiaki KASSAI, Kanagawa (JP); Keiko SEKIYA, Kanagawa (JP); Xi SHI, Cambridge, MA (US)

(73) Assignee: TAKEDA PHARMACEUTICAL COMPANY LIMITED, Osaka (JP)

(21) Appl. No.: 19/479,208

(22) PCT Filed: Apr. 26, 2024
(86) PCT No.: PCT/IB2024/054093

§ 371 Date: Oct. 27, 2025

Related U.S. Application Data
(60) Provisional application No. 63/498,773, filed on Apr. 27, 2023.
(60) Provisional application No. 63/572,039, filed on Mar. 29, 2024.

(51) Int. Cl. A61K 35/17; A61K 40/15; A61K 40/31; A61K 40/42; C07K 14/54; C07K 14/705; C07K 14/715; C12N 5/0783; C12N 15/62

(52) U.S. Cl. CPC A61K 35/17; A61K 40/15; A61K 40/31; A61K 40/4211; C07K 14/5443; C07K 14/70503; C07K 14/7155; C12N 5/0646; C12N 15/62; C07K 2319/00; C12N 2501/233; C12N 2506/11; C12N 2510/00

(57) ABSTRACT

The present disclosure relates to methods of expanding or producing CD56⁺/CD3⁻ cells comprising contacting the CD56⁺/CD3⁻ cells with an NKp46 agonist and/or a CD30 agonist.

### CROSS-REFERENCE TO RELATED APPLICATIONS

This application claims priority to and benefit of U.S. Provisional Application Nos. 63/572,039, filed Mar. 29, 2024, and 63/498,773, filed Apr. 27, 2023, each of which is hereby incorporated by reference in its entirety.

### REFERENCE TO SEQUENCE LISTING SUBMITTED ELECTRONICALLY

The content of the electronically submitted sequence listing (Name: 3817_188PC02_Seqlisting_ST26; Size: 4,409 bytes; and Date of Creation: Apr. 19, 2024) is incorporated herein by reference in its entirety.

### FIELD

The present disclosure relates to expansion of CD56+/CD3⁻ cells.

### BACKGROUND

Natural killer (NK) cells have therapeutic potential for a wide variety of human malignancies including adoptive immunotherapy for anti-cancer treatment and other indications. NK cells do not require a priming phase and are not HLA restricted, and so represent potential for an “off the shelf” potent anti-tumor response. Inducible pluripotent stem cells (iPSC)-derived NK (iNK) cells have been included in multiple clinical trials for treatment of hematological malignancies as well as for treatment of solid tumors. Because NK cells expand poorly in vitro, have limited life spans in vivo, and represent a small fraction of human peripheral blood mononuclear cells (PBMCs), obtaining sufficient cell numbers is a major obstacle in NK-cell immunotherapy. As such, there remains a need for improvement in methods for expanding NK cells and iNK cells.

### BRIEF SUMMARY

Some aspects of the present disclosure are directed to a method of expanding a CD56+/CD3− cell comprising: culturing the CD56+/CD3− cell in the presence of an NKp46 agonist, wherein the CD56+/CD3− cell comprises at least one exogenous nucleic acid molecule encoding one or more NKp46 polypeptides. Some aspects of the present disclosure are directed to a method of expanding a CD56+/CD3− cell comprising: culturing the CD56+/CD3− cell in the presence of a CD30 agonist.

In some aspects, the CD56+/CD3− cell is derived from a pluripotent stem cell. In some aspects, the method further comprises transducing the pluripotent stem cell with at least one exogenous nucleic acid molecule encoding one or more NKp46 polypeptides. In some aspects, the pluripotent stem cell is an induced pluripotent stem cell (iPSC).

In some aspects, the NKp46 agonist is an anti-NKp46 antibody. In some aspects, the CD30 agonist is an anti-CD30 antibody. In some aspects, the CD56+/CD3− cell is cultured in the presence of the NKp46 agonist and at least one cytokine. In some aspects, the at least one cytokine comprises interleukin-(IL) 2, IL-7, IL-15, IL-12, IL-18, IL-21, or any combination thereof. In some aspects, the CD56+/CD3− cell is cultured in the presence of the CD30 agonist and at least one cytokine. In some aspects, the at least one cytokine comprises interleukin-(IL) 2, IL-7, IL-15, IL-12, IL-18, IL-21, or any combination thereof.

In some aspects, the CD56+/CD3− cell further comprises at least one exogenous nucleic acid molecule encoding (i) one or more chimeric antigen receptors (CARs), and/or (ii) one or more cytokines or mimetics thereof.

In some aspects, the cytokine or the mimetic thereof is an IL-15. In some aspects, the IL-15 is linked to IL-15Ra to form a fusion protein.

Some aspects of the present disclosure are directed to a method of producing a modified CD56+/CD3− cell comprising: (A) providing an ex vivo bulk cell production comprising hematopoietic progenitor cells (HPCs), comprising at least one exogenous nucleic acid molecule encoding one or more NKp46 polypeptides, and (B) differentiating the HPCs to generate one or more CD56+/CD3− cells. In some aspects, the method further comprises: (C) culturing the CD56+/CD3− cells in the presence of an NKp46 agonist and/or a CD30 agonist.

In some aspects, the HPCs are derived from one or more pluripotent stem cells. In some aspects, the method further comprises transducing the one or more pluripotent stem cells with at least one exogenous nucleic acid molecule encoding one or more NKp46 polypeptides. In some aspects, the pluripotent stem cells are iPSCs.

In some aspects, the NKp46 agonist is an anti-NKp46 antibody. In some aspects, the CD30 agonist is an anti-CD30 antibody. In some aspects, the CD56+/CD3− cell is cultured in the presence of the NKp46 agonist and at least one cytokine. In some aspects, the at least one cytokine comprises IL-2, IL-7, IL-15, IL-12, IL-18, IL-21, or any combination thereof. In some aspects, the CD56+/CD3− cell is cultured in the presence of the CD30 agonist and at least one cytokine. In some aspects, the at least one cytokine comprises IL-2, IL-7, IL-15, IL-12, IL-18, IL-21, or any combination thereof.

In some aspects, the CD56+/CD3− cells further comprise at least one exogenous nucleic acid molecule encoding (i) one or more chimeric antigen receptors (CARs), and/or (ii) one or more cytokines or mimetics thereof.

In some aspects, the cytokine or the mimetic thereof is an IL-15. In some aspects, the IL-15 is linked to IL-15Ra to form a fusion protein.

Some aspects of the present disclosure are directed to a CD56+/CD3− cell which is obtained through a method disclosed herein.

Some aspects of the present disclosure are directed to a pharmaceutical composition comprising a CD56+/CD3− cell disclosed herein and an excipient.

Some aspects of the present disclosure are directed to a method of treating a disease or condition in a subject in need thereof, comprising administering to the subject a CD56+/CD3− cell disclosed herein or a pharmaceutical composition disclosed herein.

### BRIEF DESCRIPTION OF THE DRAWINGS/FIGURES

FIG. 1 is a graphical representation of flow cytometry results illustrating expression of the NKp46 in untransduced induced pluripotent stem cells (iPSCs) and iPSCs transduced with a construct expressing NKp46 via a CAG promoter.

FIGS. 2A and 2B are graphical representations of flow cytometry results illustrating expression of NKp46 in iNK cells differentiated from untransduced iPSC and CAG-NKp46 transduced iPSC. FIG. 2A illustrates expression of NKp46 in transduced cells and FIG. 2B confirms iNK phenotype via CD56+/CD3⁻ staining results.

FIG. 3 is a graphical representation of fold expansion of iNK cells differentiated from CAG-NKp46 transduced iPSC following a NKp46 Ab-mediated (anti-NKp46/anti-CD2) activation.

FIGS. 4A-4D are graphical representations of flow cytometry results indicating NKp46 expression and CD56 expression (FIG. 4A), CD3 expression (FIG. 4B), NKG2D expression (FIG. 4C), and CD16 expression (FIG. 4D) in iNK cells differentiated from CAG-NKp46 transduced iPSCs.

FIGS. 5A and 5B are graphical representations of the fold expansion of iNK cells following a CD30 Ab-mediated activation. FIG. 5A shows the fold expansion of iNK cells after the third expansion, and FIG. 5B shows the fold expansion of iNK cells after the fourth expansion.

FIG. 6 is a graphical representation of the in vivo persistence of iNK cells, shown by the number of human CD45+ cells in peripheral blood every week for four weeks after administration of the iNK cells.

FIGS. 7A and 7B are representations of the results of bioluminescent imaging (BLI) to assess tumor burden following administration of iNK-CD19 CAR cells. FIG. 7A is a graphical representation illustrating the quantification of the tumor burden as depicted by BLI total flux. CAR #1 is a CD19 CAR-iNK line cultured in the presence of an anti-CD3 antibody, IL-7, and IL15, as described herein; and CAR #2 is a CD19 CAR-iNK line cultured in the presence of an anti-CD16 antibody, IL-2, and IL15, as described herein. FIG. 7B is a series of photographs that illustrate the tumor burden assessed by BLI.

FIG. 7C shows the in vivo activity of CD19-mbIL15 CAR-iNKs in the Nalm6/NSG mouse model. FIG. 7C is a graphical representation of the change in body weight (percent change) of the mice.

FIG. 8 is a graphical representation of an in vitro repeat antigen stimulation assay results illustrating the tumor cell abundance measured as total RFP-positive surface area per image per well following the addition of untransduced iNK cells and iNK-mesothelin (Meso) CAR cells to each well.

FIGS. 9A and 9B are representations of the results of BLI to assess tumor burden following administration of iNK-mesothelin CAR cells in vivo. FIG. 9A is a graphical representation illustrating the quantification of the tumor burden as depicted by BLI total flux. FIG. 9B is a series of photographs that illustrate the tumor burden assessed by BLI.

FIGS. 10A and 10B are graphical representations illustrating the in vivo iNK-mesothelin CAR cell proliferation. FIG. 10A shows the percentage of live cells that are human CD45⁺ cells in mouse peripheral blood. FIG. 10B shows the absolute number of human CD45⁺ cells per μL of mouse peripheral blood.

### DETAILED DESCRIPTION

Some aspects of the present disclosure are directed to methods of expanding a CD56+/CD3− cell comprising culturing the CD56+/CD3− cell in the presence of an NKp46 agonist, wherein the CD56+/CD3− cell comprises at least one exogenous nucleic acid molecule encoding one or more NKp46 polypeptides. In some aspects, the CD56+/CD3− cell is derived from a pluripotent stem cell. In some aspects, the method further comprises transducing the pluripotent stem cell with an exogenous nucleic acid sequence encoding one or more NKp46 polypeptide.

Some aspects of the present disclosure are directed to methods of producing a modified CD56+/CD3− cell comprising: (a) providing an ex vivo bulk cell production comprising hematopoietic progenitor cells (HPCs), comprising at least one exogenous nucleic acid molecule encoding one or more NKp46 polypeptides, and (b) differentiating the HPCs to generate one or more CD56+/CD3− cells. In some aspects, the method further comprises (c) culturing the CD56+/CD3− cells in the presence of an NKp46 agonist.

Some aspects of the present disclosure are directed to methods of expanding a CD56+/CD3− cell comprising culturing the CD56+/CD3− cells in the presence of a CD30 agonist (for example, an anti-CD30 antibody).

Some aspects of the present disclosure are directed to methods of expanding a CD56+/CD3− cell comprising culturing the CD56+/CD3− cells in the presence of a CD16 agonist (for example, an anti-CD16 antibody). In some aspects, the method further comprises culturing the cells in IL-7.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

As described herein, any concentration range, percentage range, ratio range or integer range is to be understood to include the value of any integer within the recited range and, when appropriate, fractions thereof (such as one tenth and one hundredth of an integer), unless otherwise indicated.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). In addition, “or” is used to mean an open list of the components in the list. For example, “wherein X comprises A or B” means X comprises A, X comprises B, X comprises A and B, or X comprises A or B and any other components.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “activated immune cells,” “activated T cells,” and “activated NK cells” refer to, among other things, immune cells, e.g., T cells and/or NK cells, that are undergoing cell division.

An “antigen” refers to any molecule, e.g., a peptide, that provokes an immune response or is capable of being bound by a TCR. The immune response may involve antibody production, the activation of specific immunologically-competent cells, or a combination thereof. A person of skill in the art would readily understand that any macromolecule, including virtually all proteins or peptides, can serve as an antigen. An antigen can be endogenously expressed, i.e. expressed by genomic DNA, or can be recombinantly expressed. An antigen and/or an epitope can be specific to a certain tissue, such as a cancer cell, or it can be broadly expressed. In addition, fragments of larger molecules can act as antigens. In some aspects, the antigen is a hematopoietic stem cell (HSC) antigen.

An “antigen-presenting cell” or “APC,” as used herein, refers to a cell or a cell-like antigen-presenting surface that expresses one or more antigens. In some aspects, the antigen is displayed on the surface of the APC.

As used herein, the term “antigen-binding domain” refers to any protein having a binding domain that is homologous to an immunoglobulin binding domain. “Antigen-binding domain” and “antibody” further include a polypeptide comprising a framework region from an immunoglobulin protein or portions thereof that specifically binds and recognizes an antigen, and comprises at least one CDR. Use of the term “antigen-binding domain” is meant to include whole antibodies, polyclonal, monoclonal and recombinant antibodies, portions thereof, and further includes single-chain antibodies, humanized antibodies, murine antibodies, chimeric, mouse-human, mouse-primate, primate-human monoclonal antibodies, anti-idiotype antibodies, antibody constructs, such as, e.g., scFv, (scFv) 2, Fab, Fab′, and F(ab′) 2, F(ab1) 2, Fv, dAb, and Fd, disulfide-linked Fvs (dsFcs), and antibody-related polypeptides.

As used herein the term “bulk cell production” refers to using cells without cell selection/sorting by magnetic, fluorescent, chemical-based method or the like.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “autologous” refers to any material, e.g., an immune cell, nucleic acid sequence, polypeptide, or other biological material derived from the same source to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

As used herein, the term “culturing a cell population in the presence of a substance” refers, for example, to culturing a cell population in a medium containing the substance. Such culture can be, for example, culture in a medium containing the substance alone or the substance in coexistence with other differentiation inducing factors or the like. When the substance is added to the culture medium, it may be added directly to the medium, or it may be added to the medium after dissolving the substance in an appropriate solvent at the time of use. The substance can also be immobilized on a substrate or carrier surface for culture. As used herein, the terms “culturing a cell population in the presence of a substance” and “a cell population is contacted with a substance” are used interchangeably.

The term “endogenous” refers to an agent, cell, gene, polypeptide, or part thereof that grows in or originates from within an organism. In the present disclosure, an endogenous HSC is an HSC or hematopoietic stem cell originating from or within, or presently within or inside a subject.

The terms “exogenous” and “heterologous” are used interchangeably and refer to any material, e.g., an immune cell, nucleic acid sequence, gene, polypeptide, or other biological matter, derived from another individual or species, or artificially manufactured, i.e. is non-native to the gene sequence, cell, system, or subject to which it is introduced.

The term “wild-type” refers to an original, native, non-mutated, or non-truncated gene or protein sequence of an organism. A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some embodiments, the methods of the present invention can be used to reduce the tumor size of a tumor derived from, for example, the cancer comprises bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, breast cancer, prostate cancer, lung cancer (e.g., non-small cell lung cancer (NSCLC) or small cell lung cancer (SCLC)), Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. The particular cancer can be responsive to chemo- or radiation therapy or the cancer can be refractory. A refractory cancer refers to a cancer that is not amendable to surgical intervention, and the cancer is either initially unresponsive to chemo- or radiation therapy or the cancer becomes unresponsive over time.

It is understood that wherever aspects are described herein with the language “comprising,” otherwise analogous aspects described in terms of “consisting of” and/or “consisting essentially of” are also provided.

A “cytokine,” as used herein, refers to a non-antibody protein (or fusion protein comprising (i) the non-antibody protein and (ii) receptor, receptor subunit, or a portion of the receptor, etc.) that is released by one cell in response to contact with a specific antigen, wherein the cytokine interacts with a second cell to mediate a response in the second cell. A cytokine can be endogenously expressed by a cell, added to a cell in culture, administered to a subject, or any combination thereof. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, IL-21, and interferon (IFN) gamma. Examples of pro-inflammatory cytokines include, but are not limited to, IL-la, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (SICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein la (MIP-la, MIP-la), MIP-Ib (MIP-1b), gamma-induced protein 10 (IP-10), and thymus and activation regulated chemokine (TARC or CCL17).

Other examples of cytokines include, but are not limited to, chemokine (C-C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, IL-23, granulocyte colony-stimulating factor (G-CSF), leukemia inhibitory factor (LIF), oncostatin M (OSM), CD 154, lymphotoxin (LT) beta, 4-IBB ligand (4-1BBL), a proliferation-inducing ligand (APRIL), CD30L,CD70, CD153, CD178, glucocorticoid-induced TNFR-related ligand (GITRL), tumor necrosis factor superfamily member 14 (TNFSF14), OX40L, TNF-αnd ApoL-related leukocyte-expressed ligand 1 (TALL-1), or TNF-related apoptosis-inducing ligand (TRAIL). In some embodiments, cytokines include, but are not limited to, fusion protein of IL 15 and IL-15Ra.

The term “mimetic” refers to any agent which has the same function as another agent, compound, chemical, molecule, or the like. In some aspects of the present disclosure, a cytokine mimetic comprises any agent that has the same function as the particular cytokine.

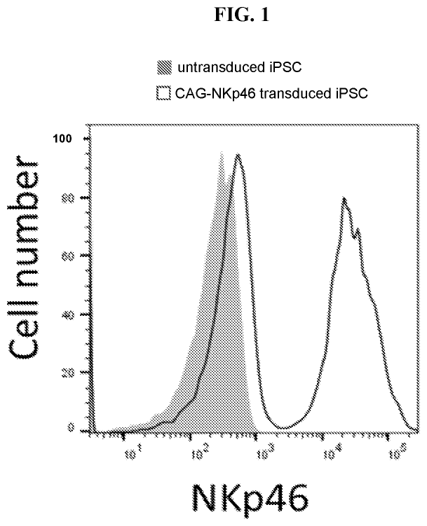

The term “engineered Autologous Cell Therapy,” which can be abbreviated as “eACT™,” also known as adoptive cell transfer, is a process by which a patient's own immune cells, e.g., T cells and/or NK cells, are collected and subsequently genetically altered to recognize and target one or more antigens expressed on the cell surface of one or more specific tumor cells or malignancies. Immune cells, e.g., T cells and/or NK cells, can be engineered to express, for example, chimeric antigen receptors (CAR) or T cell receptors (TCR). CAR positive (+) immune cells, e.g. T cells or immune cells, are engineered to express an extracellular single chain variable fragment (scFv) with specificity for a particular tumor antigen linked to an intracellular signaling part comprising a costimulatory domain and an activating domain. The costimulatory domain can be derived from, e.g., CD28, and the activating domain can be derived from, e.g., CD3-zeta (FIG. 1). In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignancies, including but not limited to NHL, CLL, and non-T cell ALL. In some aspects, the CAR scFV can bind mesothelin. Mesothelin is a protein expressed in many solid tumors, including but not limited to mesothelioma, ovarian cancer, pancreatic adenocarcinoma, and lung and uterine malignancies. Example CAR+ T cell therapies and constructs are described in U.S. Patent Publication Nos. 2013/0287748, 2014/0227237, 2014/0099309, and 2014/0050708, and these references are incorporated by reference in their entirety.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. In some aspects, an immune response refers to NK cell-mediated killing of a foreign cell, e.g., an allogeneic T cell therapy.

As used herein, the term “introducing” or “introduction” refers to expressing a heterologous polynucleotide and/or polypeptide in a cell. In some aspects, introduction is achieved by transfecting the cell with a polynucleotide of interest. In some aspects, introduction is achieved by genetically modifying the cell to express a heterologous sequence, e.g., using a gene editing tool including, but not limited to, CRISPR/Cas, CRISPR/Cas9, CRISPR/Cas12, CRISPR/Cas12a, CRISPR/Cpf1, zinc finger, TALEN, Closver-Cas or a variant thereof. In some aspects, introduction is achieved by contacting the cell with an mRNA encoding a polypeptide of interest, such that the mRNA enters into the cell or the nucleus of the cell. In some aspects, introduction comprises transfecting or transducing a cell with a polynucleotide encoding a polypeptide.

The term “lymphocyte” as used herein includes natural killer (NK) cells, T cells, NKT cells, or B cells. NK cells are a type of cytotoxic (cell toxic) lymphocyte that represent a major component of the innate immune system. NK cells reject tumors and cells infected by viruses by inducing apoptosis or programmed cell death in the target cell. NK cells are typically defined as CD3−/CD56+/CD7+ cells. In certain embodiments, NK cells may also express CD16 (CD16+) and/or NKG2D (NKG2D+). In certain embodiments, the immune cells produced using methods described herein are NK cells, characterized as CD56+/CD3− cells.

A CD56+/CD3− cell as defined herein refers to a cell that comprises functional CD56 and does not comprise functional CD3. In certain embodiments, a CD56+/CD3− cell may be an NK cell. In another example, a CD56+/CD3− cell may be a progenitor cell of an NK cell. A CD56+/CD3− cell can be prepared according to any method known in the art (see, for example, US2021/0292713 which is incorporated herein by reference).

As used herein, the term “pluripotent stem cell” or “PS cell” or “PSC” refers to a cell that is able to self-renew by dividing and developing into the three primary groups of cells that make up a human body, including ectoderm, endoderm, and mesoderm cells. Pluripotent stem cells are able to make cells from all three of these basic body layers, so they can potentially produce any cell or tissue the body needs to repair itself. This property may be referred to as pluripotency.

As used herein, the term “induced pluripotent stem cell” or “iPS cell” or “iPSC” refers to a cell that has been de-differentiated (or reprogrammed) into a more naïve, e.g., pluripotent, state. Various methods of de-differentiating a cell are known, including, but not limited to, over-expressing Oct3/4, Sox2, Klf4, and c-Myc (the “Yamanaka factors”) in the cell (see, e.g., Takahashi and Yamanaka, Cell 126:663-76 (2006)). In some aspects, the iPS cell is a pluripotent cell, e.g., capable of differentiation into a limited number of cell types. In some aspects, the iPS cell is a totipotent cell, e.g., capable of differentiation into any cell type. In some aspects, an iPS cell can be re-differentiated into a specific type of cell, e.g., an immune cell.

The term “CD3” refers to cluster of differentiation 3 which is a multimeric protein complex known as the T3 complex which serves as a T cell co-receptor and is a defining feature of T cells. Specificity of the CD3 antigen for T cells makes it a useful marker for T cells at all phases of development. CD3 is also weakly expressed by some macrophages including cells derived from the B cell lineage but is not expressed in native NK cells.

The term “CD56” refers to the neural cell adhesion molecule (NCAM) and is a member of the immunoglobulin superfamily engaged in both homophilic and heterophilic interactions. CD56 is a phenotypic marker of NK cells. Further, deficiencies and phenotypic alterations of the CD56+ immune cell fraction correlate to increased susceptibility to various infections, autoimmune, and malignant diseases.

The term “CD30,” also known as “tumor necrosis factor receptor superfamily member 8” or “TNFRSF8,” refers to a cell membrane protein of the tumor necrosis factor receptor family, and a tumor marker. CD30 is generally expressed on activated T cells, NK cells, and B cells.

The term “NKp46”, also known as CD335, refers to a receptor expressed by human NK cells that belongs to the natural cytotoxicity receptor (NCR) family. NKp46 polypeptide used in the present invention is not limited to wild-type protein as long as it is activated by the NKp46 agonist described below. To know whether such NKp46 polypeptide can be activated by the NKp46 agonist or not can be detected by confirming that downstream molecules (e.g., PI3K and ERK1/2) of NKp46 are phosphorylated. In some embodiments, the NKp46 polypeptide comprises the amino acid sequence of SEQ ID NO: 2, or an amino acid sequence at least about 75%, at least about 80%, at least about 85%, at least about 90%, at least about 95%, at least about 96%, at least about 97%, at least about 98%, or at least about 99% identical to SEQ ID NO: 2.

An exemplary nucleic acid sequence encoding NKp46 may include the nucleic acid sequence set forth in SEQ ID NO: 1 (SEQ ID NO: 1; Table 1). In some aspects, the polynucleotide comprises a nucleotide sequence having at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 1. In some aspects, the nucleic acid sequence encodes an NKp46 polypeptide comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2. In some aspects, the nucleic acid sequence encodes an NKp46 polypeptide comprising the amino acid sequence set forth in SEQ ID NO: 2.

TABLE 1
NKp46 Sequences

NKp46 Nucleic Acid Sequence
Atgtcttccacactccctgccctgctctgcgtcgggctgtgtctg
agtcagaggatcagcgcccagcagcagactctcccaaaaccgttc
atctgggccgagccccatttcatggttccaaaggaaaagcaagtg
accatctgttgccagggaaattatggggctgttgaataccagctg
cactttgaaggaagcctttttgccgtggacagaccaaaaccccct
gagcggattaacaaagtccaattctacatcccggacatgaactcc
cgcatggcagggcaatacagctgcatctatcgggttggggagctc
tggtcagagcccagcaacttgctggatctggtggtaacagaaatg
tatgacacacccaccctctcggttcatcctggacccgaagtgatc
tcgggagagaaggtgaccttctactgccgtctagacactgcaaca
agcatgttcttactgctcaaggagggaagatccagccacgtacag
cgcggatacgggaaggtccaggcggagttccccctgggccctgtg
accacagcccacagagggacataccgatgttttggctcctataac
aaccatgcctggtctttccccagtgagccagtgaagctcctggtc
acaggcgacattgagaacaccagccttgcacctgaagaccccacc
tttcctgcagacacttggggcacctaccttttaaccacagagacg
ggactccagaaagaccatgccctctgggatcacactgcccagaat
ctccttcggatgggcctggcctttctagtcctggtggctctagtg
tggttcctggttgaagactggctcagcaggaagaggactagagag
cgagccagcagagcttccacttgggaaggcaggagaaggctgaac
acacagactctttga
(SEQ ID NO: 1)
NKp46 Amino Acid Sequence
MSSTLPALLCVGLCLSQRISAQQQTLPKPFIWAEPHFMVPKEKQV
TICCQGNYGAVEYQLHFEGSLFAVDRPKPPERINKVQFYIPDMNS
RMAGQYSCIYRVGELWSEPSNLLDLVVTEMYDTPTLSVHPGPEVI
SGEKVTFYCRLDTATSMFLLLKEGRSSHVQRGYGKVQAEFPLGPV
TTAHRGTYRCFGSYNNHAWSFPSEPVKLLVTGDIENTSLAPEDPT
FPADTWGTYLLTTETGLQKDHALWDHTAQNLLRMGLAFLVLVALV
WFLVEDWLSRKRTRERASRASTWEGRRRLNTQTL
(SEQ ID NO: 2)

In some embodiments, a vector comprises any polynucleotide encoding the NKp46 is provided. The vector may be a non-viral vector such as a plasmid, or a viral vector such as an adenoviral vector, an adenovirus associated viral (AAV) vector, a lentiviral vector, and a retroviral vector. As non-limiting examples, the vector may comprise a polynucleotide of SEQ ID NO: 1. The vector may comprise a promoter upstream of the portion encoding the NKp46. An exemplary promoter used herein may be CAG promoter. In some aspects, the vector comprises a polynucleotide encoding at least one (e.g., one or more) NKp46s. In such case, the vector may comprise one or more linker sequences such as a cleavable peptide (e.g., E2A). For example, the E2A peptide may be located between two NKp46s.

In some aspects, the vector comprises a polynucleotide encoding: (i) one or more chimeric antigen receptors (CARs), and/or (ii) one or more cytokines or mimetics thereof. In some aspects, the vector comprises a polynucleotide encoding multiple (e.g., two or more) CARs and/or cytokines or mimetics thereof. In such case, the vector may comprise one or more linker sequences such as a cleavable peptide (e.g., E2A). For example, the E2A peptide may be located between CARs and/or cytokines.

The term “NKG2D” refers to a C-type lectin-like receptor expressed on NK cells, γδ T cells, CD8+ T cells, and some autoreactive or immunosuppressive CD4⁺ T cells and represents a major recognition receptor for the detection.

The term “CD16” (FcγRIIIa) refers to type I transmembrane receptor containing two extracellular Ig-like domains expressed in NK cells. CD16 function is a low affinity IgG receptor mediating antibody-dependent cellular cytotoxicity (ADCC) by NK cells.

The term “CD34” refers to an E- and P-selectin ligand on human HSPCs that binds with kinetics comparable to other known selectin ligands. CD34 is routinely used to identify and isolate human hematopoietic stem/progenitor cells (HSPCs).

The term “CD2” refers to glycoprotein that is a costimulatory receptor expressed mainly on T and NK cells.

As used herein, the term “primary NK cells” refers to an NK cell taken directly from a subject or can be the result of a culture of an NK cell isolated directly from a subject.

As used herein, the term “INK cell” or “induced NK cell” refers to an NK cell that is differentiated from an iPS cell. An iNK cell expresses CD56. In a further embodiment, such iNK cell may express CD56 and CD16. In another example, such iNK cell may express CD56 and be CD3−(CD56+/CD3−).

As used herein a “hematopoietic stem cell” or “HSC” or “hematopoietic progenitor cells” or “HPC” or “HPCs” refer to a cell that is an immature cell that can develop into all types of blood cells, including white blood cells, red blood cells, and platelets. Hematopoietic stem cells are found in the peripheral blood and the bone marrow and are also termed a “blood stem cell”.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “recombinant” or “modified” cell, as used herein, is intended to refer to a cell, e.g., an immune cell that comprises a nucleic acid that is not naturally present in the cell, and can be a cell into which a recombinant expression vector has been introduced. It should be understood that such terms are intended to refer not only to the particular subject cell but also to the progeny of such a cell. Though certain modifications can occur in succeeding generations due to either mutation or environmental influences, such progeny are still included within the scope of the term “recombinant” or “modified” as used herein.

As used herein, the terms “subject” and “patient” are used interchangeably and refer to either a human or a non-human, such as primates, mammals, and vertebrates. In particular aspects, the subject is a human.

The term “chimeric antigen receptor” or “CAR,” as used herein, refers to a recombinant fusion protein that has an antigen-specific extracellular domain coupled to an intracellular domain that directs the cell to perform a specialized function upon binding of an antigen to the extracellular domain.

A “suicide gene” or “suicide switch,” as used herein, refers to a gene that upon expression by a cell causes the cell to undergo apoptosis. Non-limiting examples of suicide genes include the inducible caspase-9 suicide gene, viral thymidine kinase, cytosine deaminases, intracellular antibody against antioxidative enzymes (AOEs), bacterial nitroreductase, other caspases, and DNase.

In some aspects, the suicide gene comprises the inducible caspase-9 suicide gene. An inducible Caspase-9 (iCasp9) “safety switch” offers a solution that allows for the removal of activated CAR T cells. The induction of iCasp9 depends on the administration of the small molecule dimerizer drug AP1903, and dimerization results in rapid induction of apoptosis in transduced cells, preferentially killing activated cells expressing high levels of transgene (see, e.g., Gargett, T, et al. Front. Pharmacol., 2014 October 28, 5:235).

In some aspects, the suicide gene comprises a viral thymidine kinase (TK). Thymidine kinase is an ATP-thymidine 5′-phosphotransferase that converts deoxythymidine into deoxythymidine 5′-monophosphate, which is further phosphorylated to deoxythymidine diphosphate and thereafter to deoxythymidine triphosphate by viral thymidine kinase and nucleoside diphosphate kinase respectively. Deoxythymidine triphosphate is incorporated into the synthesized DNA molecule by DNA polymerase. Some dNTP analogs, such as Ganciclovir (GCV), a synthetic analogue of 2′-deoxy-guanosine, have the ability to terminate the DNA synthesis upon their incorporation into synthesized DNA. Termination of synthesis triggers the apoptotic signaling cascades. While GCV is not recognized by human thymidine kinase, it is recognized as a substrate for some viral thymidine kinase, such as Herpes Simplex Virus-1 thymidine kinase (HSV-TK). As a result, a human cell expressing HSV-TK converts GCV into GCV phosphate, which is further phosphorylated and incorporated into the synthesized DNA, leading to the termination of synthesis and apoptosis. While the variant of HSV-TK is not limited, HSV-TK is, for example, TK007 (see Preuss et al., Hum Gene Ther. 2010 August; 21 (8): 929-41).

In some aspects, the suicide gene is cytosine deaminase. Cytosine deaminase hydrolyzes cytosine to uracil with the release of ammonia. In physiological conditions, the modified site is recognized by endonucleases, then the phosphodiester bond in the DNA is broken, initiating repair by incorporation of a new cytosine. However, cytosine deaminase can also convert 5-fluorocytosine into 5-fluorouracil (5-FU). Therefore, upon provision of non-toxic prodrug 5-FC, cytosine deaminase converts it into highly toxic 5-FU (a suicide inhibitor of thymidylate synthetase), leading to the inhibition of cell growth and apoptosis.

The term “inducible” refers to a response or state of activity in response to the presence of an activator or initiator. A gene or enzyme may be inducible being activated or undergoing expression only in the presence of a particular molecule.

The term “activator” or “initiator” refers to a molecule, chemical, substrate, or agent that initiates or activates activity or expression of a gene, protein, or any other biological component.

The term “CAG promoter” refers to a hybrid nucleic acid construct consisting of the cytomegalovirus (CMV) enhancer fused to the chicken β-actin (CAG) promoter.

The term “promoter” is a sequence of DNA to which proteins bind to initiate transcription of RNA from the DNA downstream of the promoter.

An “NKp46 agonist” is an agent, whether a chemical or biological agent that activates, upregulates, or stimulates NKp46. The NKp46 agonist can be used alone or in combination with two or more NKp46 agonists. In some aspects, the NKp46 agonist comprises an anti-NKp46 antibody that can activate, upregulate, or stimulate NKp46 (In this specification, such anti-NKp46 antibody that can activate, upregulate, or stimulate NKp46 is sometimes referred as “anti-NKp46 antibody” or “anti-NKp46 agonistic antibody”). Such anti-NKp46 antibody may include, but not be limited to, Clone 9E2, 4k12, n1D9, B-N40, B-L46, 900, 2B11A3, 8F24 and 195314 (R&D systems, Biolegend, Miltenyi biotec, etc.), NK Cell Activation/Expansion Kit human (Miltenyi biotec) and Cloudz human NK cell expansion kit (R&D systems). Such antibodies may be functional fragments thereof, such as Fd, Fv, Fab, F(ab′), F(ab) 2, F(ab′) 2, single chain Fv (scFv), diabody, triabody, F(ab) 2, F(ab′2), F(ab′2), single-chain Fv (scFv), diabody, triabody, tetrabody and minibody. The antibodies can be derived from animals such as mouse, rat, cow, rabbit, goat, sheep, guinea pig, etc. The isotypes of antibodies are not restricted, and the isotypes include IgG (IgG1, IgG2, IgG3, IgG4), IgA, IgD, IgE and IgM.

A “CD30 agonist” is an agent, whether a chemical or biological agent, that activates, upregulates, or stimulates CD30. CD30 agonist can be used alone or in combination with two or more CD30 agonists. In some aspects, the CD30 agonist comprises an anti-CD30 antibody that can activate, upregulate, or stimulate CD30 (i.e., an “anti-CD30 antibody” or “anti-CD30 agonistic antibody”). Such an anti-CD30 antibody can be, but is not limited to, Clone #81316 (R&D systems). Such antibodies can be functional fragments thereof, such as Fd, Fv, Fab, F(ab′), F(ab) 2, F(ab′) 2, single chain Fv (scFv), diabody, triabody, F(ab) 2, F(ab′2), F(ab′2), single-chain Fv (scFv), diabody, triabody, tetrabody and minibody. The antibodies can be derived from animals such as mouse, rat, cow, rabbit, goat, sheep, guinea pig, etc. The isotypes of antibodies are not restricted, and the isotypes include IgG (IgG1, IgG2, IgG3, IgG4), IgA, IgD, IgE and IgM.

The antibodies may be monoclonal or polyclonal antibodies, preferably monoclonal antibodies, and the antibodies may also be humanized antibodies, chimeric antibodies, and multispecific antibodies (e.g., bispecific antibodies).

Such antibodies can be produced by known methods, for example, by constructing an expression vector containing nucleic acid encoding the antibody, culturing a transformant to which the nucleic acid is introduced, or culturing a hybridoma producing the antibody.

The term “caspase inhibitors” refers to any agent, chemical or biological, that inhibit caspases. Caspases may be controlled in several ways including processing and activation regulation by molecules such as FADD, APAF-1, Bcl-2 family members, FLIP, and IAPs. Active caspases can be controlled by a variety of inhibitors some of which include products of both viral and cellular genes, as well as artificial caspase inhibitors developed for research and pharmaceutical agents (Ekert, et al. Cell Death. Differ, November 1999, 6 (11): 1081-6).

A “medium” or “culture medium” as used herein refers to an aqueous solution of nutrients which can be used for growing cells over a prolonged period of time, i.e., a liquid used in a cell culture.

A NK related disease may comprise any condition or disease related to abnormal NK function. Such conditions or diseases may include, but not be limited to, cancer, viral infections including pneumonia and other viruses such as the Herpes virus and HIV, asthma, type I diabetes, rheumatoid arthritis, systemic lupus erythematosus, inflammatory bowel disease (ulcerative colitis and Crohn's disease), celiac disease, dermatomyositis, Graves disease, Hashimoto thyroiditis, myasthenia gravis, psoriasis, scleroderma, Sjogren's syndrome, and multiple sclerosis.

As used herein, the term “treating” or “treatment” of a disease or condition refers to executing a protocol, which may include administering one or more therapies to a patient, in an effort to alleviate signs or symptoms of the disease. In some aspects, a treatment decreases the rate of disease progression, ameliorates or palliates the disease state, and/or facilitates remission or improved prognosis. Alleviation can occur prior to signs or symptoms of the disease or condition appearing, as well as after their appearance. Thus, in some aspects, “treating” or “treatment” includes “preventing” or “prevention” of a disease or an undesirable condition. However, “treating” or “treatment” does not require complete alleviation of all signs and/or symptoms, does not require a cure, and specifically includes protocols that have only a marginal effect on the patient.

In various aspects, a subject in need thereof may be treated for a disease or for alleviating symptoms associated with a disease (e.g., a cancer) using a population of modified cells described herein. In some aspects, the modified cells are NK or iNK cells that are transduced with a CAR or a TCR and/or are modified to exhibit reduced expression of endogenous MHC class I and MHC class II HLAs genes in combination with expression or increased expression of certain heterologous genes in the cell, such that the modified cells are less susceptible to killing by the subject's immune system when such cells are administered to the subject.

As used herein, the terms “ug”, “uL” and “uM” are used interchangeably with “μg”, “μL”, and “μM,” respectively.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. As described herein, any concentration range, percentage range, ratio range or integer range is to be understood to include the value of any integer within the recited range and, when appropriate, fractions thereof (such as one-tenth and one-hundredth of an integer), unless otherwise indicated.

Abbreviations used herein are defined throughout the present disclosure. Various aspects of the disclosure are described in further detail in the following subsections.

Various aspects described herein are described in further detail in the following subsections.

### II. Compositions of the Disclosure

Some aspects of the present disclosure are directed to a method of expanding a CD56+/CD3− cell comprising: culturing the CD56+/CD3− cell in the presence of an NKp46 agonist, wherein the CD56+/CD3− cell comprises at least one exogenous nucleic acid molecule encoding one or more NKp46 polypeptides. Some aspects of the present disclosure are directed to a method of expanding a CD56+/CD3− cell comprising: culturing the CD56+/CD3− cell in the presence of a CD30 agonist. In some aspects, the CD56+/CD3− cell is derived from a pluripotent stem cell.

Some aspects of the present disclosure are directed to a method of producing a modified CD56+/CD3− cell comprising: (A) providing an ex vivo bulk cell production comprising hematopoietic progenitor cells (HPCs), comprising at least one exogenous nucleic acid molecule encoding one or more NKp46 polypeptides, and (B) differentiating the HPCs to generate one or more CD56+/CD3− cells. In some aspects, the method further comprises (C) culturing the CD56+/CD3− cells in presence of an NKp46 agonist and/or a CD30 agonist. In some aspects, the HPCs are derived from one or more pluripotent stem cells.

### II.A. Methods of the Disclosure

### II.A.1. NKp46 Agonist

Some aspects of the present disclosure are directed to methods of producing and/or expanding CD56+/CD3− cells. In some aspects, the method comprises culturing the CD56+/CD3− cell in presence of an NKp46 agonist. Any NKp46 agonist can be used in the methods disclosed herein. In some aspects, the NKp46 agonist is a polypeptide. In some aspects, the NKp46 agonist is an antibody or an antigen-binding portion thereof that specifically binds NKp46 (an “anti-NKp46 antibody”). In some aspects, the NKp46 agonist comprises a small molecule.

In some aspects, the NKp46 agonist comprises an anti-NKp46 antibody. Any anti-NKp46 antibody can be used in the methods disclosed herein. In some aspects, the CD56+/CD3− cell is contacted with about 100 ng/ml to about 10 μg/ml anti-NKp46 antibody. In some aspects, the CD56+/CD3− cell is contacted with about 500 ng/ml to about 5 μg/ml anti-NKp46 antibody. In some aspects, the CD56+/CD3− cell is contacted with about 100 ng/ml, about 150 ng/ml, about 200 ng/ml, about 250 ng/ml, about 300 ng/ml, about 350 ng/ml, about 400 ng/ml, about 450 ng/ml, about 500 ng/ml, about 600 ng/ml, about 700 ng/ml, about 800 ng/ml, about 900 ng/ml, about 1 μg/ml, about 2 ug/ml, about 3 ug/ml, about 4 ug/ml, about 5 ug/ml, about 6 ug/ml, about 7 ug/ml, about 8 ug/ml, about 9 ug/ml or about 10 ug/ml anti-NKp46 antibody. In some aspects, the CD56+/CD3− cell is contacted with about 1 μg/ml anti-NKp46 antibody.

In some aspects, the CD56+/CD3− cell is further contacted with at least one cytokine. In some aspects, the at least one cytokine is selected from IL-2, IL-7, IL-12, IL-15, IL-18, and IL-21. In some aspects, the CD56+/CD3− cell is contacted with an NKp46 agonist and IL-2. In some aspects, the CD56+/CD3− cell is contacted with an NKp46 agonist and IL-7. In some aspects, the CD56+/CD3− cell is contacted with an NKp46 agonist and IL-12. In some aspects, the CD56+/CD3− cell is contacted with an NKp46 agonist and IL-15. In some aspects, the CD56+/CD3− cell is contacted with an NKp46 agonist and IL-18. In some aspects, the CD56+/CD3− cell is contacted with an NKp46 agonist and IL-21.

In some aspects, the CD56+/CD3− cell is contacted with an NKp46 agonist and IL-2 and IL-7. In some aspects, the CD56+/CD3− cell is contacted with an NKp46 agonist and IL-2 and IL-12. In some aspects, the CD56+/CD3− cell is contacted with an NKp46 agonist and IL-2 and IL-15. In some aspects, the CD56+/CD3− cell is contacted with an NKp46 agonist and IL-2 and IL-18. In some aspects, the CD56+/CD3− cell is contacted with an NKp46 agonist and IL-2 and IL-21. In some aspects, the CD56+/CD3− cell is contacted with an NKp46 agonist and IL-2, IL-10, and IL-15. In some aspects, the CD56+/CD3− cell is contacted with an NKp46 agonist and IL-2, IL-7, IL-12, IL-15, and IL-21. In some aspects, the CD56+/CD3− cell is contacted with an NKp46 agonist (preferably, anti-NKp46 agonistic antibody) and IL-2, IL-7, IL-12, IL-15, IL-18, and IL-21.

Some aspects of the present disclosure are directed to a method of producing an iNK cell, the method comprising: (A) providing an ex vivo bulk cell production comprising hematopoietic progenitor cells (HPCs), comprising at least one exogenous nucleic acid molecule encoding one or more NKp46 polypeptides, and (B) differentiating the HPCs to generate one or more CD56+/CD3− cells. In some aspects, the method further comprises (C) culturing the CD56+/CD3− cells in presence of an NKp46 agonist. In some aspects the CD56+/CD3− cells are contacted with an NKp46 agonist. In some aspects, the CD56+/CD3−cells are contacted with an NKp46 agonist for about 1 day. In some aspects, the CD56+/CD3−cells are contacted with an NKp46 agonist for about 2 days. In some aspects, the CD56+/CD3−cells are contacted with an NKp46 agonist for about 3 days. In some aspects, the CD56+/CD3−cells are contacted with an NKp46 agonist for about 4 days. In some aspects, the CD56+/CD3−cells are contacted with an NKp46 agonist for about 5 days. In some aspects, the CD56+/CD3−cells are contacted with an NKp46 agonist for about 6 days. In some aspects, the CD56+/CD3−cells are contacted with an NKp46 agonist for about 7 days. In some aspects, the CD56+/CD3−cells are contacted with an NKp46 agonist for about 8 days. In some aspects, the CD56+/CD3−cells are contacted with an NKp46 agonist for about 9 days. In some aspects, the CD56+/CD3−cells are contacted with an NKp46 agonist for about 10 days. In some aspects, the CD56+/CD3−cells are contacted with an NKp46 agonist for about 11 days. In some aspects, the CD56+/CD3−cells are contacted with an NKp46 agonist for about 12 days. In some aspects, the CD56+/CD3−cells are contacted with an NKp46 agonist for about 13 days. In some aspects, the CD56+/CD3−cells are contacted with an NKp46 agonist for about 14 days. In some aspects, the CD56+/CD3−cells are contacted with an NKp46 agonist for about 15 days. In some aspects, the CD56+/CD3−cells are contacted with an NKp46 agonist for about 16 days. In some aspects, the CD56+/CD3−cells are contacted with an NKp46 agonist for about 17 days. In some aspects, the CD56+/CD3−cells are contacted with an NKp46 agonist for about 18 days. In some aspects, the CD56+/CD3−cells are contacted with an NKp46 agonist for about 19 days. In some aspects, the CD56+/CD3−cells are contacted with an NKp46 agonist for about 20 days. In some aspects, the CD56+/CD3−cells are contacted with an NKp46 agonist for about 21 days.

In some aspects, the CD56+/CD3− cells are contacted with (i) an NKp46 agonist, IL-2, IL-7, IL-15, and one or more of IL-21, IL12, and IL-18 for a first culture; and then the CD56+/CD3− cells are contacted with (ii) an NKp46 agonist, IL-2, IL-7, and IL-15 for a second culture. In some aspects, the first culture continues for about 1 day. In some aspects, the first culture continues for about 2 days. In some aspects, the first culture continues for about 3 days. In some aspects, the first culture continues for about 4 days. In some aspects, the first culture continues for about 5 days. In some aspects, the first culture continues for about 6 days. In some aspects, the first culture continues for about 7 days. In some aspects, the first culture continues for about 8 days. In some aspects, the first culture continues for about 9 days. In some aspects, the first culture continues for about 10 days. In some aspects, the first culture continues for about 11 days. In some aspects, the first culture continues for about 12 days. In some aspects, the first culture continues for about 13 days. In some aspects, the first culture continues for about 14 days. In some aspects, the second culture continues for about 1 day. In some aspects, the second culture continues for about 2 days. In some aspects, the second culture continues for about 3 days. In some aspects, the second culture continues for about 4 days. In some aspects, the second culture continues for about 5 days. In some aspects, the second culture continues for about 6 days. In some aspects, the second culture continues for about 7 days. In some aspects, the second culture continues for about 8 days. In some aspects, the second culture continues for about 9 days. In some aspects, the second culture continues for about 10 days. In some aspects, the second culture continues for about 11 days. In some aspects, the second culture continues for about 12 days. In some aspects, the second culture continues for about 13 days. In some aspects, the second culture continues for about 14 days.

In some aspects, the second culture is repeated to increase the number of cells generated. In some aspects, the second culture is conducted two times. In some aspects, the second culture is conducted three times. In some aspects, the second culture is conducted four times. In some aspects, the second culture is conducted five times. In some aspects, the second culture is conducted six times. In some aspects, the second culture is conducted seven times. In some aspects, the second culture is conducted eight times. In some aspects, the second culture is conducted nine times. In some aspects, the second culture is conducted ten times. In some aspects, the second culture is conducted more than ten times.

### II.A.2, CD30 Agonist

In some aspects, the method comprises culturing the CD56+/CD3− cell in presence of CD30 agonist. Any CD30 agonist can be used in the methods disclosed herein. In some aspects, the CD30 agonist is a polypeptide. In some aspects, the CD30 agonist is an antibody or an antigen-binding portion thereof that specifically binds CD30 (an “anti-CD30 antibody”). In some aspects, the CD30 agonist comprises a small molecule.

In some aspects, the CD30 agonist comprises an anti-CD30 antibody. Any anti-CD30 antibody can be used in the methods disclosed herein. In some aspects, the CD56+/CD3−cell is contacted with about 100 ng/ml to about 10 μg/ml anti-CD30 antibody. In some aspects, the CD56+/CD3− cell is contacted with about 250 ng/ml to about 2.5 μg/ml anti-CD30 antibody. In some aspects, the CD56+/CD3− cell is contacted with about 100 ng/ml, about 150 ng/ml, about 200 ng/ml, about 250 ng/ml, about 300 ng/ml, about 350 ng/ml, about 400 ng/ml, about 450 ng/ml, about 500 ng/ml, about 600 ng/ml, about 700 ng/ml, about 800 ng/ml, about 900 ng/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml anti-CD30 antibody. In some aspects, the CD56+/CD3− cell is contacted with about 200 ng/ml anti-CD30 antibody. In some aspects, the CD56+/CD3− cell is contacted with about 225 ng/ml anti-CD30 antibody. In some aspects, the CD56+/CD3− cell is contacted with about 250 ng/ml anti-CD30 antibody. In some aspects, the CD56+/CD3− cell is contacted with about 275 ng/ml anti-CD30 antibody. In some aspects, the CD56+/CD3− cell is contacted with about 300 ng/ml anti-CD30 antibody. In some aspects, the CD56+/CD3− cell is contacted with about 325 ng/ml anti-CD30 antibody. In some aspects, the CD56+/CD3− cell is contacted with about 350 ng/ml anti-CD30 antibody. In some aspects, the CD56+/CD3− cell is contacted with about 375 ng/ml anti-CD30 antibody. In some aspects, the CD56+/CD3− cell is contacted with about 400 ng/ml anti-CD30 antibody. In some aspects, the CD56+/CD3− cell is contacted with about 500 ng/ml anti-CD30 antibody. In some aspects, the CD56+/CD3− cell is contacted with about 600 ng/ml anti-CD30 antibody. In some aspects, the CD56+/CD3− cell is contacted with about 700 ng/ml anti-CD30 antibody. In some aspects, the CD56+/CD3− cell is contacted with about 800 ng/ml anti-CD30 antibody. In some aspects, the CD56+/CD3− cell is contacted with about 900 ng/ml anti-CD30 antibody. In some aspects, the CD56+/CD3− cell is contacted with about 1 μg/ml anti-CD30 antibody.

In some aspects, the CD56+/CD3− cell is further contacted with at least one cytokine. In some aspects, the at least one cytokine is selected from IL-2, IL-7, IL-12, IL-15, IL-18, and IL-21. In some aspects, the CD56+/CD3− cell is contacted with a CD30 agonist and IL-2. In some aspects, the CD56+/CD3− cell is contacted with a CD30 agonist and IL-7. In some aspects, the CD56+/CD3− cell is contacted with a CD30 agonist and IL-12. In some aspects, the CD56+/CD3− cell is contacted with a CD30 agonist and IL-15. In some aspects, the CD56+/CD3− cell is contacted with a CD30 agonist and IL-18. In some aspects, the CD56+/CD3− cell is contacted with a CD30 agonist and IL-21.

In some aspects, the CD56+/CD3− cell is contacted with a CD30 agonist and IL-2 and IL-7. In some aspects, the CD56+/CD3− cell is contacted with a CD30 agonist and IL-2 and IL-12. In some aspects, the CD56+/CD3− cell is contacted with a CD30 agonist and IL-2 and IL-15. In some aspects, the CD56+/CD3− cell is contacted with a CD30 agonist and IL-2 and IL-18. In some aspects, the CD56+/CD3− cell is contacted with a CD30 agonist and IL-2 and IL-21. In some aspects, the CD56+/CD3− cell is contacted with a CD30 agonist and IL-2, IL-10, and IL-15. In some aspects, the CD56+/CD3− cell is contacted with a CD30 agonist and IL-2, IL-7, IL-12, IL-15, and IL-21. In some aspects, the CD56+/CD3− cell is contacted with a CD30 agonist (e.g., an anti-CD30 agonistic antibody) and IL-2, IL-7, IL-12, IL-15, IL-18, and IL-21.

Some aspects of the present disclosure are directed to a method of producing an iNK cell, the method comprising: (A) providing an ex vivo bulk cell production comprising hematopoietic progenitor cells (HPCs), comprising at least one exogenous nucleic acid molecule encoding one or more NKp46 polypeptides, and (B) differentiating the HPCs to generate one or more CD56+/CD3− cells. In some aspects, the method further comprises (C) culturing the CD56+/CD3− cells in presence of a CD30 agonist. In some aspects the CD56+/CD3− cells are contacted with a CD30 agonist. In some aspects, the CD56+/CD3− cells are contacted with a CD30 agonist for about 1 day. In some aspects, the CD56+/CD3− cells are contacted with a CD30 agonist for about 2 days. In some aspects, the CD56+/CD3− cells are contacted with a CD30 agonist for about 3 days. In some aspects, the CD56+/CD3− cells are contacted with a CD30 agonist for about 4 days. In some aspects, the CD56+/CD3− cells are contacted with a CD30 agonist for about 5 days. In some aspects, the CD56+/CD3− cells are contacted with a CD30 agonist for about 6 days. In some aspects, the CD56+/CD3− cells are contacted with a CD30 agonist for about 7 days. In some aspects, the CD56+/CD3− cells are contacted with a CD30 agonist for about 8 days. In some aspects, the CD56+/CD3− cells are contacted with a CD30 agonist for about 9 days. In some aspects, the CD56+/CD3− cells are contacted with a CD30 agonist for about 10 days. In some aspects, the CD56+/CD3− cells are contacted with a CD30 agonist for about 11 days. In some aspects, the CD56+/CD3− cells are contacted with a CD30 agonist for about 12 days. In some aspects, the CD56+/CD3− cells are contacted with a CD30 agonist for about 13 days. In some aspects, the CD56+/CD3− cells are contacted with a CD30 agonist for about 14 days. In some aspects, the CD56+/CD3− cells are contacted with a CD30 agonist for about 15 days. In some aspects, the CD56+/CD3− cells are contacted with a CD30 agonist for about 16 days. In some aspects, the CD56+/CD3− cells are contacted with a CD30 agonist for about 17 days. In some aspects, the CD56+/CD3− cells are contacted with a CD30 agonist for about 18 days. In some aspects, the CD56+/CD3− cells are contacted with a CD30 agonist for about 19 days. In some aspects, the CD56+/CD3− cells are contacted with a CD30 agonist for about 20 days. In some aspects, the CD56+/CD3− cells are contacted with a CD30 agonist for about 21 days.

Some aspects of the present disclosure are directed to a method of producing an iNK cell, the method comprising: (A) providing an ex vivo bulk cell production comprising hematopoietic progenitor cells (HPCs), and (B) differentiating the HPCs to generate one or more CD56+/CD3− cells. In some aspects, the method further comprises (C) culturing the CD56+/CD3− cells in presence of a CD30 agonist. In some aspects the CD56+/CD3− cells are contacted with a CD30 agonist. In some aspects, the CD56+/CD3− cells are contacted with a CD30 agonist for about 1 day. In some aspects, the CD56+/CD3− cells are contacted with a CD30 agonist for about 2 days. In some aspects, the CD56+/CD3− cells are contacted with a CD30 agonist for about 3 days. In some aspects, the CD56+/CD3− cells are contacted with a CD30 agonist for about 4 days. In some aspects, the CD56+/CD3− cells are contacted with a CD30 agonist for about 5 days. In some aspects, the CD56+/CD3− cells are contacted with a CD30 agonist for about 6 days. In some aspects, the CD56+/CD3− cells are contacted with a CD30 agonist for about 7 days. In some aspects, the CD56+/CD3− cells are contacted with a CD30 agonist for about 8 days. In some aspects, the CD56+/CD3− cells are contacted with a CD30 agonist for about 9 days. In some aspects, the CD56+/CD3− cells are contacted with a CD30 agonist for about 10 days. In some aspects, the CD56+/CD3− cells are contacted with a CD30 agonist for about 11 days. In some aspects, the CD56+/CD3− cells are contacted with a CD30 agonist for about 12 days. In some aspects, the CD56+/CD3− cells are contacted with a CD30 agonist for about 13 days. In some aspects, the CD56+/CD3− cells are contacted with a CD30 agonist for about 14 days. In some aspects, the CD56+/CD3− cells are contacted with a CD30 agonist for about 15 days. In some aspects, the CD56+/CD3− cells are contacted with a CD30 agonist for about 16 days. In some aspects, the CD56+/CD3− cells are contacted with a CD30 agonist for about 17 days. In some aspects, the CD56+/CD3− cells are contacted with a CD30 agonist for about 18 days. In some aspects, the CD56+/CD3− cells are contacted with a CD30 agonist for about 19 days. In some aspects, the CD56+/CD3− cells are contacted with a CD30 agonist for about 20 days. In some aspects, the CD56+/CD3− cells are contacted with a CD30 agonist for about 21 days.

In some aspects, the CD56+/CD3− cells are contacted with (i) a CD30 agonist, IL-2, IL-7, IL-15, and one or more of IL-21, IL12, and IL-18 for a first culture; and then the CD56+/CD3− cells are contacted with (ii) a CD30 agonist, IL-2, IL-7, and IL-15 for a second culture. In some aspects, the first culture continues for about 1 day. In some aspects, the first culture continues for about 2 days. In some aspects, the first culture continues for about 3 days. In some aspects, the first culture continues for about 4 days. In some aspects, the first culture continues for about 5 days. In some aspects, the first culture continues for about 6 days. In some aspects, the first culture continues for about 7 days. In some aspects, the first culture continues for about 8 days. In some aspects, the first culture continues for about 9 days. In some aspects, the first culture continues for about 10 days. In some aspects, the first culture continues for about 11 days. In some aspects, the first culture continues for about 12 days. In some aspects, the first culture continues for about 13 days. In some aspects, the first culture continues for about 14 days. In some aspects, the second culture continues for about 1 day. In some aspects, the second culture continues for about 2 days. In some aspects, the second culture continues for about 3 days. In some aspects, the second culture continues for about 4 days. In some aspects, the second culture continues for about 5 days. In some aspects, the second culture continues for about 6 days. In some aspects, the second culture continues for about 7 days. In some aspects, the second culture continues for about 8 days. In some aspects, the second culture continues for about 9 days. In some aspects, the second culture continues for about 10 days. In some aspects, the second culture continues for about 11 days. In some aspects, the second culture continues for about 12 days. In some aspects, the second culture continues for about 13 days. In some aspects, the second culture continues for about 14 days. In some aspects, the second culture continues for about 15 days.

In some aspects, the second culture is repeated to increase the number of cells generated. In some aspects, the second culture is conducted twice. In some aspects, the second culture is conducted three times. In some aspects, the second culture is conducted four times. In some aspects, the second culture is conducted five times. In some aspects, the second culture is conducted six times. In some aspects, the second culture is conducted seven times. In some aspects, the second culture is conducted eight times. In some aspects, the second culture is conducted nine times. In some aspects, the second culture is conducted ten times. In some aspects, the second culture is conducted more than ten times.

In some aspects, the method comprises culturing the CD56+/CD3− cell in presence of an NKp46 agonist and a CD30 agonist. In some aspects, the NKp46 agonist comprises an anti-NKp46 antibody, and the CD30 agonist comprises an anti-CD30 antibody. Any anti-NKp46 antibody or anti-CD30 antibody can be used in the methods disclosed herein. In some aspects, the CD56+/CD3− cell is expanded with an anti-NKp46 antibody. In some aspects, the anti-NKp46 antibody is plate-bound. In some aspects, the CD56+/CD3− cell is contacted with about 100 ng/ml to about 10 μg/ml anti-NKp46 antibody and/or CD30 agonist. In some aspects, the CD56+/CD3− cell is contacted with about 500 ng/ml to about 5 μg/ml anti-NKp46 antibody and/or CD30 agonist. In some aspects, the CD56+/CD3− cell is contacted with about 100 ng/ml, about 150 ng/ml, about 200 ng/ml, about 250 ng/ml, about 300 ng/ml, about 350 ng/ml, about 400 ng/ml, about 450 ng/ml, about 500 ng/ml, about 600 ng/ml, about 700 ng/ml, about 800 ng/ml, about 900 ng/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml or about 10 μg/ml anti-NKp46 antibody and/or CD30 agonist. In some aspects, the CD56+/CD3− cell is contacted with about 1 μg/ml anti-NKp46 antibody and/or CD30 agonist. In some aspects, the CD56+/CD3−cell is contacted with plate bound NKp46 antibody and about 300 ng/ml anti-CD30 antibody.

In some aspects, the CD56+/CD3− cell is further contacted with at least one cytokine. In some aspects, the at least one cytokine is selected from IL-2, IL-7, IL-12, IL-15, IL-18, and IL-21. In some aspects, the CD56+/CD3− cell is contacted with an NKp46 agonist, a CD30 agonist, and IL-2. In some aspects, the CD56+/CD3− cell is contacted with an NKp46 agonist, a CD30 agonist, and IL-7. In some aspects, the CD56+/CD3− cell is contacted with an NKp46 agonist, a CD30 agonist, and IL-12. In some aspects, the CD56+/CD3− cell is contacted with an NKp46 agonist, a CD30 agonist, and IL-15. In some aspects, the CD56+/CD3− cell is contacted with an NKp46 agonist, a CD30 agonist, and IL-18. In some aspects, the CD56+/CD3− cell is contacted with an NKp46 agonist, a CD30 agonist, and IL-21.

In some aspects, the CD56+/CD3− cell is contacted with an NKp46 agonist, a CD30 agonist, and IL-2 and IL-7. In some aspects, the CD56+/CD3− cell is contacted with an NKp46 agonist, a CD30 agonist, and IL-2 and IL-12. In some aspects, the CD56+/CD3− cell is contacted with an NKp46 agonist, a CD30 agonist, and IL-2 and IL-15. In some aspects, the CD56+/CD3− cell is contacted with an NKp46 agonist, a CD30 agonist, and IL-2 and IL-18. In some aspects, the CD56+/CD3− cell is contacted with an NKp46 agonist, a CD30 agonist, and IL-2 and IL-21. In some aspects, the CD56+/CD3− cell is contacted with an NKp46 agonist, a CD30 agonist, and IL-2, IL-10, and IL-15. In some aspects, the CD56+/CD3− cell is contacted with an NKp46 agonist, a CD30 agonist, and IL-2, IL-7, IL-12, IL-15, and IL-21. In some aspects, the CD56+/CD3− cell is contacted with an NKp46 agonist (preferably, anti-NKp46 agonistic antibody), a CD30 agonist (preferably, anti-CD30 agonistic antibody), and IL-2, IL-7, IL-12, IL-15, IL-18, and IL-21.

Some aspects of the present disclosure are directed to a method of producing an iNK cell, the method comprising: (A) providing an ex vivo bulk cell production comprising hematopoietic progenitor cells (HPCs), comprising at least one exogenous nucleic acid molecule encoding one or more NKp46 polypeptides, and (B) differentiating the HPCs to generate one or more CD56+/CD3− cells. In some aspects, the method further comprises (C) culturing the CD56+/CD3− cells in presence of an NKp46 agonist and a CD30 agonist. In some aspects the CD56+/CD3− cells are contacted with an NKp46 agonist and a CD30 agonist. In some aspects, the CD56+/CD3− cells are contacted with an NKp46 agonist and a CD30 agonist for about 1 day. In some aspects, the CD56+/CD3− cells are contacted with an NKp46 agonist and a CD30 agonist for about 2 days. In some aspects, the CD56+/CD3− cells are contacted with an NKp46 agonist and a CD30 agonist for about 3 days. In some aspects, the CD56+/CD3−cells are contacted with an NKp46 agonist and a CD30 agonist for about 4 days. In some aspects, the CD56+/CD3− cells are contacted with an NKp46 agonist and a CD30 agonist for about 5 days. In some aspects, the CD56+/CD3− cells are contacted with an NKp46 agonist and a CD30 agonist for about 6 days. In some aspects, the CD56+/CD3− cells are contacted with an NKp46 agonist and a CD30 agonist for about 7 days. In some aspects, the CD56+/CD3−cells are contacted with an NKp46 agonist and a CD30 agonist for about 8 days. In some aspects, the CD56+/CD3− cells are contacted with an NKp46 agonist and a CD30 agonist for about 9 days. In some aspects, the CD56+/CD3− cells are contacted with an NKp46 agonist and a CD30 agonist for about 10 days. In some aspects, the CD56+/CD3− cells are contacted with an NKp46 agonist and a CD30 agonist for about 11 days. In some aspects, the CD56+/CD3−cells are contacted with an NKp46 agonist and a CD30 agonist for about 12 days. In some aspects, the CD56+/CD3− cells are contacted with an NKp46 agonist and a CD30 agonist for about 13 days. In some aspects, the CD56+/CD3− cells are contacted with an NKp46 agonist and a CD30 agonist for about 14 days. In some aspects, the CD56+/CD3− cells are contacted with an NKp46 agonist and a CD30 agonist for about 15 days. In some aspects, the CD56+/CD3− cells are contacted with an NKp46 agonist and a CD30 agonist for about 16 days. In some aspects, the CD56+/CD3− cells are contacted with an NKp46 agonist and a CD30 agonist for about 17 days. In some aspects, the CD56+/CD3− cells are contacted with an NKp46 agonist and a CD30 agonist for about 18 days. In some aspects, the CD56+/CD3− cells are contacted with an NKp46 agonist and a CD30 agonist for about 19 days. In some aspects, the CD56+/CD3− cells are contacted with an NKp46 agonist and a CD30 agonist for about 20 days. In some aspects, the CD56+/CD3− cells are contacted with an NKp46 agonist and a CD30 agonist for about 21 days.

In some aspects, the CD56+/CD3− cells are contacted with (i) an NKp46 agonist, a CD30 agonist, IL-2, IL-7, IL-15, and one or more of IL-21, IL12, and IL-18 for a first culture; and then the CD56+/CD3− cells are contacted with (ii) an NKp46 agonist, a CD30 agonist, IL-2, IL-7, and IL-15 for a second culture. In some aspects, the first culture continues for about 1 day. In some aspects, the first culture continues for about 2 days. In some aspects, the first culture continues for about 3 days. In some aspects, the first culture continues for about 4 days. In some aspects, the first culture continues for about 5 days. In some aspects, the first culture continues for about 6 days. In some aspects, the first culture continues for about 7 days. In some aspects, the first culture continues for about 8 days. In some aspects, the first culture continues for about 9 days. In some aspects, the first culture continues for about 10 days. In some aspects, the first culture continues for about 11 days. In some aspects, the first culture continues for about 12 days. In some aspects, the first culture continues for about 13 days. In some aspects, the first culture continues for about 14 days. In some aspects, the second culture continues for about 1 day. In some aspects, the second culture continues for about 2 days. In some aspects, the second culture continues for about 3 days. In some aspects, the second culture continues for about 4 days. In some aspects, the second culture continues for about 5 days. In some aspects, the second culture continues for about 6 days. In some aspects, the second culture continues for about 7 days. In some aspects, the second culture continues for about 8 days. In some aspects, the second culture continues for about 9 days. In some aspects, the second culture continues for about 10 days. In some aspects, the second culture continues for about 11 days. In some aspects, the second culture continues for about 12 days. In some aspects, the second culture continues for about 13 days. In some aspects, the second culture continues for about 14 days. In some aspects, the second culture continues for about 15 days.

In some aspects, the second culture is repeated to increase the number of cells generated. In some aspects, the second culture is conducted twice. In some aspects, the second culture is conducted three times. In some aspects, the second culture is conducted four times. In some aspects, the second culture is conducted five times. In some aspects, the second culture is conducted six times. In some aspects, the second culture is conducted seven times. In some aspects, the second culture is conducted eight times. In some aspects, the second culture is conducted nine times. In some aspects, the second culture is conducted ten times. In some aspects, the second culture is conducted more than ten times.

### II.A.3. Culture Reagents

In some aspects, the CD56+/CD3− cell is contacted with about 0.1 to about 100 ng/ml IL-2. In some aspects, the CD56+/CD3− cell is contacted with about 1 to about 100 ng/ml, about 1 to about 90 ng/ml, about 1 to about 80 ng/ml, about 1 to about 70 ng/ml, about 1 to about 60 ng/ml, about 1 to about 50 ng/ml, about 1 to about 40 ng/ml, about 1 to about 30 ng/ml, about 1 to about 20 ng/ml, or about 1 to about 10 ng/ml IL-2. In some aspects, the CD56+/CD3− cell is contacted with about 1 to about 20 ng/ml IL-2. In some aspects, the CD56+/CD3− cell is contacted with about 1 to about 10 ng/ml IL-2. In some aspects, the CD56+/CD3− cell is contacted with about 10 to about 20 ng/ml IL-2. In some aspects, the CD56+/CD3− cell is contacted with about 5 to about 15 ng/ml IL-2.

In some aspects, the CD56+/CD3− cell is contacted with about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 30 ng/ml, about 40 ng/ml, about 50 ng/ml, about 60 ng/ml, about 70 ng/ml, about 80 ng/ml, about 90 ng/ml, or about 100 ng/ml IL-2. In some aspects, the CD56+/CD3− cell is contacted with about 10 ng/ml IL-2.

In some aspects, the CD56+/CD3− cell is contacted with about 0.1 to about 100 ng/ml IL-7. In some aspects, the CD56+/CD3− cell is contacted with about 1 to about 100 ng/ml, about 1 to about 90 ng/ml, about 1 to about 80 ng/ml, about 1 to about 70 ng/ml, about 1 to about 60 ng/ml, about 1 to about 50 ng/ml, about 1 to about 40 ng/ml, about 1 to about 30 ng/ml, about 1 to about 20 ng/ml, or about 1 to about 10 ng/ml IL-7. In some aspects, the CD56+/CD3− cell is contacted with about 1 to about 20 ng/ml IL-7. In some aspects, the CD56+/CD3− cell is contacted with about 1 to about 10 ng/ml IL-7. In some aspects, the CD56+/CD3− cell is contacted with about 10 to about 20 ng/ml IL-7. In some aspects, the CD56+/CD3− cell is contacted with about 5 to about 15 ng/ml IL-7.

In some aspects, the CD56+/CD3− cell is contacted with about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 30 ng/ml, about 40 ng/ml, about 50 ng/ml, about 60 ng/ml, about 70 ng/ml, about 80 ng/ml, about 90 ng/ml, or about 100 ng/ml IL-7. In some aspects, the CD56+/CD3− cell is contacted with about 10 ng/ml IL-7.

In some aspects, the CD56+/CD3− cell is contacted with about 0.1 to about 100 ng/ml IL-15. In some aspects, the CD56+/CD3− cell is contacted with about 1 to about 100 ng/ml, about 1 to about 90 ng/ml, about 1 to about 80 ng/ml, about 1 to about 70 ng/ml, about 1 to about 60 ng/ml, about 1 to about 50 ng/ml, about 1 to about 40 ng/ml, about 1 to about 30 ng/ml, about 1 to about 20 ng/ml, or about 1 to about 10 ng/ml IL-15. In some aspects, the CD56+/CD3− cell is contacted with about 1 to about 20 ng/ml IL-15. In some aspects, the CD56+/CD3− cell is contacted with about 1 to about 10 ng/ml IL-15. In some aspects, the CD56+/CD3− cell is contacted with about 10 to about 20 ng/ml IL-15. In some aspects, the CD56+/CD3− cell is contacted with about 5 to about 15 ng/ml IL-15.

In some aspects, the CD56+/CD3− cell is contacted with about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 30 ng/ml, about 40 ng/ml, about 50 ng/ml, about 60 ng/ml, about 70 ng/ml, about 80 ng/ml, about 90 ng/ml, or about 100 ng/ml IL-15. In some aspects, the CD56+/CD3− cell is contacted with about 10 ng/ml IL-15.

In some aspects, the CD56+/CD3− cell is contacted with about 0.1 to about 100 ng/ml IL-21. In some aspects, the CD56+/CD3− cell is contacted with about 1 to about 100 ng/ml, about 1 to about 90 ng/ml, about 1 to about 80 ng/ml, about 1 to about 70 ng/ml, about 1 to about 60 ng/ml, about 1 to about 50 ng/ml, about 1 to about 40 ng/ml, about 1 to about 30 ng/ml, about 1 to about 20 ng/ml, or about 10 to about 20 ng/ml IL-21. In some aspects, the CD56+/CD3− cell is contacted with about 10 to about 30 ng/ml IL-21. In some aspects, the CD56+/CD3− cell is contacted with about 10 to about 20 ng/ml IL-21. In some aspects, the CD56+/CD3− cell is contacted with about 20 to about 30 ng/ml IL-21. In some aspects, the CD56+/CD3− cell is contacted with about 15 to about 25 ng/ml IL-21.

In some aspects, the CD56+/CD3− cell is contacted with about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 40 ng/ml, about 50 ng/ml, about 60 ng/ml, about 70 ng/ml, about 80 ng/ml, about 90 ng/ml, or about 100 ng/ml IL-21. In some aspects, the CD56+/CD3− cell is contacted with about 20 ng/ml IL-21.

In some aspects, the CD56+/CD3− cell is contacted with about 0.1 to about 100 ng/ml IL-12. In some aspects, the CD56+/CD3− cell is contacted with about 1 to about 100 ng/ml, about 1 to about 90 ng/ml, about 1 to about 80 ng/ml, about 1 to about 70 ng/ml, about 1 to about 60 ng/ml, about 1 to about 50 ng/ml, about 10 to about 90 ng/ml, about 20 to about 80 ng/ml, about 30 to about 70 ng/ml, or about 40 to about 60 ng/ml IL-12. In some aspects, the CD56+/CD3− cell is contacted with about 40 to about 60 ng/ml IL-12. In some aspects, the CD56+/CD3− cell is contacted with about 40 to about 50 ng/ml IL-12. In some aspects, the CD56+/CD3− cell is contacted with about 50 to about 60 ng/ml IL-12. In some aspects, the CD56+/CD3− cell is contacted with about 45 to about 55 ng/ml IL-12.

In some aspects, the CD56+/CD3− cell is contacted with about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, about 51 ng/ml, about 52 ng/ml, about 53 ng/ml, about 54 ng/ml, about 55 ng/ml, about 56 ng/ml, about 57 ng/ml, about 58 ng/ml, about 59 ng/ml, about 30 ng/ml, about 40 ng/ml, about 50 ng/ml, about 60 ng/ml, about 70 ng/ml, about 80 ng/ml, about 90 ng/ml, or about 400 ng/ml IL-12. In some aspects, the CD56+/CD3− cell is contacted with about 50 ng/ml IL-12.

In some aspects, the CD56+/CD3− cell is contacted with about 0.1 to about 100 ng/ml IL-18. In some aspects, the CD56+/CD3− cell is contacted with about 1 to about 100 ng/ml, about 1 to about 90 ng/ml, about 1 to about 80 ng/ml, about 1 to about 70 ng/ml, about 1 to about 60 ng/ml, about 1 to about 50 ng/ml, about 10 to about 90 ng/ml, about 20 to about 80 ng/ml, about 30 to about 70 ng/ml, or about 40 to about 60 ng/ml IL-18. In some aspects, the CD56+/CD3− cell is contacted with about 40 to about 60 ng/ml IL-18. In some aspects, the CD56+/CD3− cell is contacted with about 40 to about 50 ng/ml IL-18. In some aspects, the CD56+/CD3− cell is contacted with about 50 to about 60 ng/ml IL-18. In some aspects, the CD56+/CD3− cell is contacted with about 45 to about 55 ng/ml IL-18.

In some aspects, the CD56+/CD3− cell is contacted with about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, about 51 ng/ml, about 52 ng/ml, about 53 ng/ml, about 54 ng/ml, about 55 ng/ml, about 56 ng/ml, about 57 ng/ml, about 58 ng/ml, about 59 ng/ml, about 30 ng/ml, about 40 ng/ml, about 50 ng/ml, about 60 ng/ml, about 70 ng/ml, about 80 ng/ml, about 90 ng/ml, or about 400 ng/ml IL-18. In some aspects, the CD56+/CD3− cell is contacted with about 50 ng/ml IL-18.

In some aspects, the CD56+/CD3− cell is further contacted with a caspase inhibitor. Any caspase inhibitor can be used in the methods disclosed herein. In some aspects, the caspase inhibitor comprises Z-VAD-FMK. In some aspects, the CD56+/CD3− cell is contacted with about 1 to about 100 μM Z-VAD-FMK. In some aspects, the CD56+/CD3− cell is contacted with about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, or about 100 μM Z-VAD-FMK. In some aspects, the CD56+/CD3− cell is contacted with about 10 μM Z-VAD-FMK.

In some aspects, the CD56+/CD3− cell is expanded in a closed system.

In some aspects, the method comprises expanding the CD56+/CD3− cells for at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days.

In some aspects, the CD56+/CD3− cells are subjected to the first culture for about 4 days, and then the CD56+/CD3− cells are subjected to the second culture for about 10 days. In some aspects, the CD56+/CD3− cells are subjected to the first culture for about 3 days, and then the CD56+/CD3− cells are subjected to the second culture for about 10 days. In some aspects, the CD56+/CD3− cells are subjected to the first culture for about 2 days, and then the CD56+/CD3− cells are subjected to the second culture for about 10 days. In some aspects, the CD56+/CD3− cells are subjected to the first culture for about 4 days, and then the CD56+/CD3−cells are subjected to the second culture for about 9 days. In some aspects, the CD56+/CD3−cells are subjected to the first culture for about 4 days, and then the CD56+/CD3− cells are subjected to the second culture for about 8 days. In some aspects, the CD56+/CD3− cells are subjected to the first culture for about 4 days, and then the CD56+/CD3− cells are subjected to the second culture for about 7 days. In some aspects, the CD56+/CD3− cells are subjected to the first culture for about 4 days, and then the CD56+/CD3− cells are subjected to the second culture for about 11 days. In some aspects, the CD56+/CD3− cells are subjected to the first culture for about 4 days, and then the CD56+/CD3− cells are subjected to the second culture for about 12 days. In some aspects, the CD56+/CD3− cells are subjected to the first culture for about 4 days, and then the CD56+/CD3− cells are subjected to the second culture for about 13 days. In some aspects, the CD56+/CD3− cells are subjected to the first culture for about 4 days, and then the CD56+/CD3− cells are subjected to the second culture for about 14 days.

### II.A.4. Novel Methods of Manufacturing INK Cells

Some aspects of the present disclosure are directed to methods of producing and/or expanding CD56+/CD3− cells. In some aspects, the method comprises culturing the CD56+/CD3− cell in presence of a CD16 agonist. Any CD16 agonist can be used in the methods disclosed herein. In some aspects, the CD16 agonist is a polypeptide. In some aspects, the CD16 agonist is an antibody or an antigen-binding portion thereof that specifically binds CD16 (an “anti-CD16 antibody”). In some aspects, the CD16 agonist comprises a small molecule.

In some aspects, the CD16 agonist comprises an anti-CD16 antibody. Any anti-CD16 antibody can be used in the methods disclosed herein. In some aspects, the CD56+/CD3−cell is contacted with a culture vessel coated with anti-CD16 antibody. In some aspects, the culture vessel is coated with about 0.1 μg/cm² to about 1 μg/cm² anti-CD16 antibody. In some aspects, the culture vessel is coated with about 0.4 μg/cm² to about 0.8 μg/cm² anti-CD16 antibody. In some aspects, the culture vessel is coated with about 0.5 μg/cm² to about 0.7 μg/cm² anti-CD16 antibody. In some aspects, the culture vessel is coated with about 0.6 μg/cm² anti-CD16 antibody. In some aspects, the culture vessel is coated with about 0.1 μg/cm², 0.2 μg/cm², 0.3, ug/cm², 0.4 μg/cm², 0.5 μg/cm², 0.6 μg/cm², 0.7 μg/cm², 0.8 μg/cm², 0.9 μg/cm², 1 μg/cm², 1.5 μg/cm², or 2 μg/cm² anti-CD16 antibody. In some aspects, the culture vessel is coated with about 0.3 μg/cm² anti-CD16 antibody. In some aspects, the culture vessel is coated with about 0.4 μg/cm² anti-CD16 antibody. In some aspects, the culture vessel is coated with about 0.5 μg/cm² anti-CD16 antibody. In some aspects, the culture vessel is coated with about 0.6 μg/cm² anti-CD16 antibody. In some aspects, the culture vessel is coated with about 0.7 μg/cm² anti-CD16 antibody. In some aspects, the culture vessel is coated with about 0.8 μg/cm² anti-CD16 antibody. In some aspects, the culture vessel is coated with about 0.9 μg/cm² anti-CD16 antibody.

In some aspects, the CD56+/CD3− cell is further contacted with at least one cytokine. In some aspects, the at least one cytokine is selected from IL-2, IL-7, IL-12, IL-15, IL-18, and IL-21. In some aspects, the CD56+/CD3− cell is contacted with a CD16 agonist and IL-2. In some aspects, the CD56+/CD3− cell is contacted with a CD16 agonist and IL-7. In some aspects, the CD56+/CD3− cell is contacted with a CD16 agonist and IL-15. In some aspects, the CD56+/CD3− cell is contacted with a CD16 agonist and IL-18. In some aspects, the CD56+/CD3− cell is contacted with a CD16 agonist and IL-21.

In some aspects, the CD56+/CD3− cell is contacted with a CD16 agonist and IL-7 and/or IL-2. In some aspects, the CD56+/CD3− cell is contacted with a CD16 agonist and IL-7 and IL-15. In some aspects, the CD56+/CD3− cell is contacted with a CD16 agonist and IL-7 and IL-18. In some aspects, the CD56+/CD3− cell is contacted with a CD16 agonist and IL-7 and IL-21. In some aspects, the CD56+/CD3− cell is contacted with a CD16 agonist and IL-7, IL-15, and IL-18. In some aspects, the CD56+/CD3− cell is contacted with a CD16 agonist (e.g., an anti-CD16 agonistic antibody) and IL-7, IL-15, IL-18, and IL-21.

Some aspects of the present disclosure are directed to a method of producing an iNK cell, the method comprising: (A) providing an ex vivo bulk cell production comprising hematopoietic progenitor cells (HPCs), and (B) differentiating the HPCs to generate one or more CD4− cells. In some aspects, the method further comprises (C) transduction of the NK cells. In some aspects, the method of step (C) comprises differentiating the CD4− cells to generate one or more NK cells, activating and expanding the NK cells, and gammaretrovirus transduction of the NK cells. In some aspects, the method of step (C) comprises activating and expanding the NK cells and gammaretrovirus transduction of the NK cells.

In some aspects, the method comprises culturing the CD56+/CD3− cells in presence of a CD16 agonist. In some aspects the CD56+/CD3− cells are contacted with a CD16 agonist. In some aspects, the CD56+/CD3− cells are contacted with a CD16 agonist for about 1 day. In some aspects, the CD56+/CD3− cells are contacted with a CD16 agonist for about 2 days. In some aspects, the CD56+/CD3− cells are contacted with a CD16 agonist for about 3 days. In some aspects, the CD56+/CD3− cells are contacted with a CD16 agonist for about 4 days. In some aspects, the CD56+/CD3− cells are contacted with a CD16 agonist for about 5 days. In some aspects, the CD56+/CD3− cells are contacted with a CD16 agonist for about 6 days. In some aspects, the CD56+/CD3− cells are contacted with a CD16 agonist for about 7 days. In some aspects, the CD56+/CD3− cells are contacted with a CD16 agonist for about 8 days. In some aspects, the CD56+/CD3− cells are contacted with a CD16 agonist for about 9 days. In some aspects, the CD56+/CD3− cells are contacted with a CD16 agonist for about 10 days. In some aspects, the CD56+/CD3− cells are contacted with a CD16 agonist for about 11 days. In some aspects, the CD56+/CD3− cells are contacted with a CD16 agonist for about 12 days. In some aspects, the CD56+/CD3− cells are contacted with a CD16 agonist for about 13 days. In some aspects, the CD56+/CD3− cells are contacted with a CD16 agonist for about 14 days. In some aspects, the CD56+/CD3− cells are contacted with a CD16 agonist for about 15 days. In some aspects, the CD56+/CD3− cells are contacted with a CD16 agonist for about 16 days. In some aspects, the CD56+/CD3− cells are contacted with a CD16 agonist for about 17 days. In some aspects, the CD56+/CD3− cells are contacted with a CD16 agonist for about 18 days. In some aspects, the CD56+/CD3− cells are contacted with a CD16 agonist for about 19 days. In some aspects, the CD56+/CD3− cells are contacted with a CD16 agonist for about 20 days. In some aspects, the CD56+/CD3− cells are contacted with a CD16 agonist for about 21 days.

In some aspects, the CD56+/CD3− cells are contacted with (i) a CD16 agonist, IL-7, IL-15, IL-18, and IL-21 for a first culture; and then the CD56+/CD3− cells are contacted with (ii) IL-7 and IL-15 for a second culture. In some aspects, the first culture continues for about 1 day. In some aspects, the first culture continues for about 2 days. In some aspects, the first culture continues for about 3 days. In some aspects, the first culture continues for about 4 days. In some aspects, the first culture continues for about 5 days. In some aspects, the first culture continues for about 6 days. In some aspects, the first culture continues for about 7 days. In some aspects, the first culture continues for about 8 days. In some aspects, the first culture continues for about 9 days. In some aspects, the first culture continues for about 10 days. In some aspects, the first culture continues for about 11 days. In some aspects, the first culture continues for about 12 days. In some aspects, the first culture continues for about 13 days. In some aspects, the first culture continues for about 14 days. In some aspects, the second culture continues for about 1 day. In some aspects, the second culture continues for about 2 days. In some aspects, the second culture continues for about 3 days. In some aspects, the second culture continues for about 4 days. In some aspects, the second culture continues for about 5 days. In some aspects, the second culture continues for about 6 days. In some aspects, the second culture continues for about 7 days. In some aspects, the second culture continues for about 8 days. In some aspects, the second culture continues for about 9 days. In some aspects, the second culture continues for about 10 days. In some aspects, the second culture continues for about 11 days. In some aspects, the second culture continues for about 12 days. In some aspects, the second culture continues for about 13 days. In some aspects, the second culture continues for about 14 days.

### II.B. Cells of the Disclosure

Any cell type can be used in the compositions and methods disclosed herein. In some aspects, the cell is an immune cell. In some aspects, the cell is a pluripotent stem cell (PSC). In some aspects, the cell is an induced pluripotent stem cell (iPSC). In some aspects, the cell is an embryonic stem cell (ESC). In some aspects, the cell is an immune cell selected from a T cell, an NK cell, an NKT cell, or a tumor-infiltrating lymphocyte.

In some aspects, the cell is a cell differentiated from an iPSC. In some aspects, the cell is an immune cell differentiated from an iPSC. In some aspects, the cell is a CD56+/CD3−cell differentiated from an iPSC. In some aspects, the cell is an NK cell differentiated from an iPSC. In some aspects, the cell is an NKT cell differentiated from an iPSC.

In some aspects, the cell is a CD56+/CD3− cell differentiated from an iPSC, wherein the iPSC is transduced with at least one exogenous nucleic acid molecule encoding one or more NKp46 polypeptides. In some aspects, the iPSC cell is transduced before the iPSC cell is differentiated to a CD56+/CD3− cell. As such, some aspects of the present disclosure are directed to methods of expanding a CD56+/CD3− cell, wherein the cell comprises at least one exogenous nucleic acid molecule encoding one or more NKp46 polypeptides.

In some aspects, the CD56+/CD3− cell further comprises at least one exogenous nucleic acid molecule encoding one or more chimeric antigen receptors (CARs). In some aspects, the CD56+/CD3− cell further comprises at least one exogenous nucleic acid molecule encoding one or more cytokines or mimetics thereof. In some aspects, the CD56+/CD3− cell further comprises (i) at least one exogenous nucleic acid molecule encoding a chimeric antigen receptor (CAR), and (ii) at least one exogenous nucleic acid molecule encoding a cytokine or a mimetic thereof. In some aspects, the CD56+/CD3− cell is differentiated from an iPSC, wherein the iPSC is transduced with (i) at least one exogenous nucleic acid molecule encoding a chimeric antigen receptor (CAR), (ii) at least one exogenous nucleic acid molecule encoding a cytokine or a mimetic thereof, (iii) suicide switch, or (iv) any combination of (i)-(iii).

In some aspects, the at least one exogenous nucleic acid molecule encoding a cytokine or a mimetic thereof encodes an IL-15 polypeptide. In some aspects, the IL-15 polypeptide comprises an amino acids sequence having at least about 70%, at least about 75%, at least about 80%, at least about 85%, at least about 90%, at least about 95%, at least about 96%, at least about 97%, at least about 98%, at least about 99% sequence identity to the amino acid sequence of human IL-15 (UniProt P40933). In some aspects, the IL-15 polypeptide comprises human IL-15 (UniProt P40933). In some aspects, the at least one exogenous nucleic acid molecule encoding a cytokine or a mimetic thereof encodes an IL-15 polypeptide mimetic. In some aspects, the at least one exogenous nucleic acid molecule encoding a cytokine or a mimetic thereof encodes a IL-15 polypeptide comprising IL-15 linked to IL-15Ra to form a fusion protein (IL-15Ra/IL-15 complex) (for example, IL-15Ra/IL-15 complex disclosed in US2021/0292713 which is incorporated herein by reference).

### II.C. Methods of Treatment

Some aspects of the present disclosure are directed to methods of treating a disease or condition in a subject in need thereof comprising administering to the subject a composition disclosed herein. In some aspects, the method comprises administering a modified or engineered cell disclosed herein. In some aspects, the method comprises administering a population of cells disclosed herein. In some aspects, the method comprises administering a composition comprising a CD56+/CD3− cell or iNK cell, or a CD56+/CD3− cell or iNK cell included with a pharmaceutically acceptable carrier.

In some aspects, the disease or condition comprises a NK-related disease.

In some aspects, the disease or condition comprises a cancer, e.g., the subject is afflicted with a cancer. In some aspects, the cancer comprises bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. In some aspects, the cancer is locally advanced. In some aspects, the cancer is metastatic. In some aspects, the cancer is refractory. In some aspects, the cancer is relapsed. In some aspects, the cancer is refractory or relapsed following one or more prior anti-cancer therapy. In some aspects, the one or more prior anti-cancer therapy comprises a standard of care therapy.

In some aspects, the compositions disclosed herein are administered in combination with an additional anti-cancer therapy. In some aspects, the additional anti-cancer therapy comprises a chemotherapy, an immunotherapy, a radiotherapy, a surgery, or any combination thereof. In some aspects, the additional anti-cancer therapy comprises a chemotherapy. In some aspects, the additional anti-cancer therapy comprises an immune-checkpoint inhibitor. In some aspects, the additional anti-cancer therapy comprises a PD-1 antagonist, a PD-L1 antagonist, a CTLA-4 antagonist, a LAG-3 antagonist, a GITR antagonist, or any combination thereof. In some aspects, the anti-cancer therapy comprises an antibody or antigen-binding portion thereof the specifically binds and inhibits PD-1. In some aspects, the anti-cancer therapy comprises an antibody or antigen-binding portion thereof the specifically binds and inhibits PD-L1.

In some aspects, the method further comprises pretreating the subject prior to administering the population of immune cells. In some aspects, the subject is administered a chemotherapy prior to administering the population of immune cells. In some aspects, the subject is administered an immuno-depleting chemotherapy prior to administering the population of immune cells. In some aspects, the immuno-depleting chemotherapy comprises cyclophosphamide, fludarabine, or both.

In some aspects, the method comprises administering to the subject (i) the population of expanded cells and (ii) a cytokine. In some aspects, the cytokine comprises IL-2, an analog thereof, a variant thereof, or a fragment thereof.

In some aspects, the cells of the present disclosure are administered to a subject at a dose of at least about 1×10⁶ cells, at least about 2×10⁶ cells, at least about 3×10⁶ cells, at least about 4×10⁶ cells, at least about 5×10⁶ cells, 1×10⁷ cells, at least about 2×10⁷ cells, at least about 3×10⁷ cells, at least about 4×10⁷ cells, at least about 5×10⁷ cells, 1×10⁸ cells, at least about 2×10⁸ cells, at least about 3×10⁸ cells, at least about 4×10⁸ cells, at least about 5×10⁸ cells, 1×10⁹ cells, at least about 2×10⁹ cells, at least about 3×10⁹ cells, at least about 4×10⁹ cells, or at least about 5×10⁹ cells.

The cells of the present disclosure have low toxicity and thus can be safely administered in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

In certain embodiments of the invention, methods of the present invention for clinical aspects are combined with other agents effective in the treatment of the disease or condition comprises a NK-related disease as described above.

### II.D. Cell Therapy Products

Some aspects of the present disclosure are directed to a population of cells comprising a plurality of modified cells disclosed herein. Some aspects of the present disclosure are directed to a cell therapy comprising a population of modified cells disclosed herein. In some aspects, the population of cells is cryopreserved. Any method of cryopreservation of cells, e.g., immune cells, can be used in the methods and compositions disclosed herein. In some aspects, the cells are cryopreserved in the presence of DMSO. In some aspects, the cell therapy is cryopreserved to facilitate shipment of the cells.

The cell therapy and/or population of cells disclosed herein can be further formulated with one or more excipient. Any excipient that can preserve the cells can be used in the methods and compositions disclosed herein. In some aspects, the cell therapy and/or population of cells is formulated with one or more excipient that allows for cryopreservation of the cells, e.g., DMSO.

In some aspects, the population of cells comprises a plurality of modified cells disclosed herein and one or more additional cell. In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the population of cells comprise a plurality of modified cells disclosed herein. In some aspects, at least about 10% of the cells in the population of cells comprise a plurality of modified cells disclosed herein. In some aspects, at least about 20% of the cells in the population of cells comprise a plurality of modified cells disclosed herein. In some aspects, at least about 25% of the cells in the population of cells comprise a plurality of modified cells disclosed herein. In some aspects, at least about 30% of the cells in the population of cells comprise a plurality of modified cells disclosed herein. In some aspects, at least about 40% of the cells in the population of cells comprise a plurality of modified cells disclosed herein. In some aspects, at least about 50% of the cells in the population of cells comprise a plurality of modified cells disclosed herein. In some aspects, at least about 60% of the cells in the population of cells comprise a plurality of modified cells disclosed herein. In some aspects, at least about 65% of the cells in the population of cells comprise a plurality of modified cells disclosed herein. In some aspects, at least about 70% of the cells in the population of cells comprise a plurality of modified cells disclosed herein. In some aspects, at least about 75% of the cells in the population of cells comprise a plurality of modified cells disclosed herein. In some aspects, at least about 80% of the cells in the population of cells comprise a plurality of modified cells disclosed herein. In some aspects, at least about 90% of the cells in the population of cells comprise a plurality of modified cells disclosed herein.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986)); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1

### Generation of NKp46-Transduced Induced Pluripotent Stem Cells

To generate NKp46-transduced induced pluripotent stem cells (iPSCs), the iPSCs, strain Ff101s04, were seeded at 1×10⁴ cells/well in iMatrix511 coated 24-well plate with StemFit AK03N medium with included 50 μM Y-27632, a selective p160ROCK inhibitor. The next day, culture medium was replaced with StemFit AK03N and lentivirus vectors expressing the NKp46 gene (SEQ ID NO: 1) under control of a CAG promoter were infected to the iPSCs to produce FfI01s04/CAG-NKp46 cells. Proliferated FfI01s04/CAG-NKp46 cells were stained with anti-NKp46 antibody conjugated with anti-allophycocyanin (APC, BioLegend) and analyzed by flow cytometry to quantitate the number of cells expressing NKp46. The number of cells expressing NKp46 in untransduced iPSCs and CAG-NKp46 transduced iPSCs is shown in FIG. 1.

### Example 2

Differentiation of iNK Cells from NKp46-Transduced iPSC

To differentiate invariant natural killer (iNK) cells from NKp46-transduced iPSCs, FfI01s04/NKp46 cells were dispersed in StemFit AK03N medium and seeded at 5×10⁵ cells/well in an ultra-low adhesion-treated 6 well plate under low-oxygen (5% O₂) conditions on Day 0. The StemFit AK03N medium included 10 μM CHIR99021, a GSK-3 inhibitor and 10 μM Y-27632. The next day (Day 1), the cells were dispersed in human pluripotent stem cell (HPC) induction medium (Invitrogen containing bone morphogenic protein 4 (BMP4) (50 ng/ml, R&D systems), vascular endothelial growth factor (VEGF, 50 ng/ml, R&D systems), basic fibroblast growth factor (bFGF, 50 ng/ml, Wako), and ascorbic acid 2-phosphate (50 mg/ml, Sigma Aldrich). The HPC induction medium included StemPro34 supplemented with L-glutamine (4 mM, Sigma Aldrich), penicillin (100 U/ml, Sigma Aldrich), streptomycin (100 mg/ml, Sigma Aldrich), human insulin (10 mg/ml, Invitrogen), human transferrin (5.5 mg/ml, Invitrogen), sodium selenite (6.7 ng/ml, Invitrogen), Glutamax (1×, Invitrogen) and a-monothioglycerol (0.4 mM, Invitrogen). On Day 2, SB431542 inhibitor of activin receptor-like kinase receptors (at 6 μM in medium, FUJIFILM Wako Pure Chemical Corporation) was added directly to the culture medium, and the cells were cultured for 2 days. On Day 4, cells were redispersed in another HPC induction medium containing VEGF(50 ng/ml), bFGF(50 ng/ml), SCF(50 ng/ml, R&D systems), and ascorbic acid 2-phosphate (50 mg/ml), and cultured for another 3 days. On Day 7, cells were exposed to another HPC induction medium containing VEGF(50 ng/ml), bFGF(50 ng/ml), SCF(50 ng/ml), ascorbic acid 2-phosphate (50 mg/ml), TPO (30 ng/ml, Peprotech) and Flt3L (10 ng/ml, Peprotech), and cultured using this medium for additional 7 days. The medium was changed every 2-3 days during this 7-day culture period.

At Day 14, cells were harvested and CD34 positive cells were isolated by CD34-Microbeads (Miltenyi Biotech) and seeded in 10 cm dishes at 1.16×10⁵ cells/dish. Each 10 cm dish was coated with rh-DLL4/Fc chimera (Sino Biological) and RetroNectin (Takara Bio Inc.). The medium was changed every 2-3 days during this culture period. MEM a medium (Gibco, Thermo Fisher Scientific) supplemented with 15% fetal bovine serum (FBS, Hyclone), 1× Glutamax, 4 mM L-glutamine, 100 U/ml penicillin, 100 mg/ml streptomycin, 55 μM 2-mercaptoethanol, 50 mg/ml ascorbic acid 2-phosphate, 10 mg/ml human insulin, 5.5 mg/ml human transferrin, 6.7 ng/ml sodium selenite, 50 ng/ml SCF, 50 ng/ml IL-7 (Peprotech), 50 ng/ml Flt3L, 100 ng/ml TPO, 15 μM SB203580 (Tocris), 30 nM stromal cell-derived factor-1 alpha (SDF-1 a, Peprotech) was used as the medium to culture these cells. On Day 21, the cells were harvested and seeded at 1×10⁶ cells/dish in new 15 cm dishes freshly coated with hDLL4/RetroNectin and all cells were harvested on Day 35.

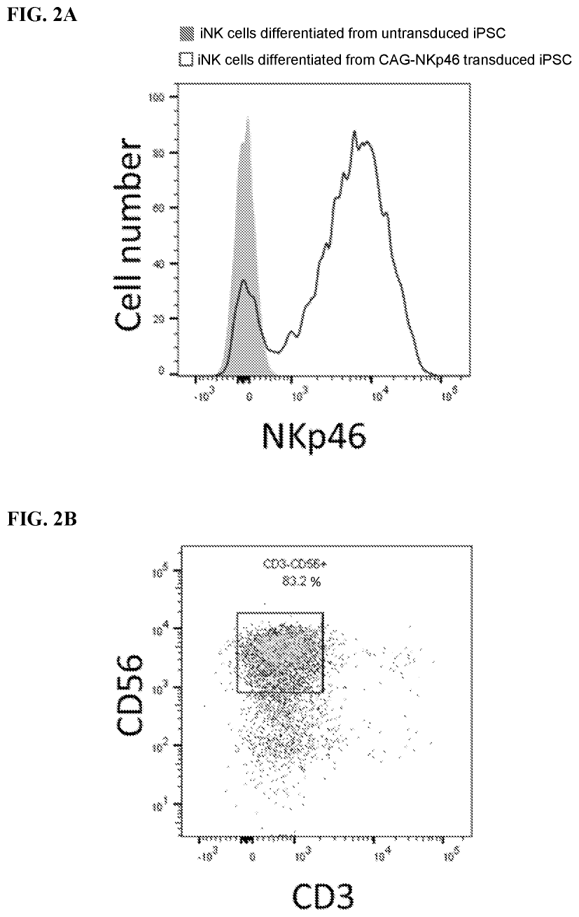

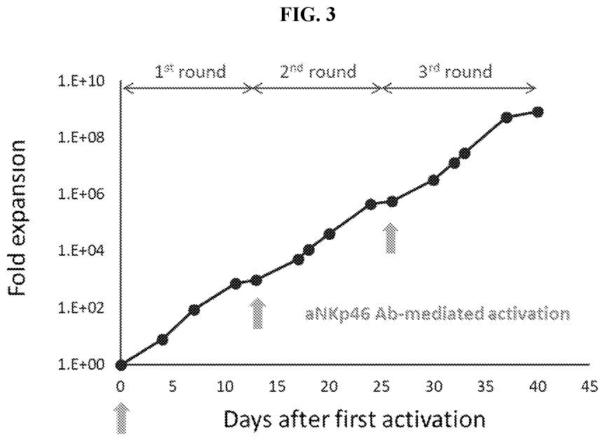

On Day 35, the harvested cells were seeded at 5×10⁵ cells/well in 48-well plate in MEM a medium supplemented with 15% FBS, 4 mM L-glutamine, 100 U/ml penicillin, 100 mg/ml streptomycin, 50 mg/ml ascorbic acid 2-phosphate, 10 mg/ml human insulin, 5.5 mg/ml human transferrin, 6.7 ng/ml sodium selenite, 10 ng/ml IL-2 (Peprotech), and 10 ng/ml IL-7. On Day 39, half of medium in each wells were changed and on Day 42 all cells were harvested as differentiated iNK cells. iNK cells were stained with antibodies as listed in Table 2, and analyzed number of cells expressing NKp46 and cell number of the CD56+/CD3− phenotype cells by flow cytometer as shown in FIGS. 2A and 2B, respectively. Results indicate that the cells expressing NKp46 make up a good percentage of the population of cells and CD56+/CD3−comprise about 83.2% of the cell population.

TABLE 2
Stains used to detect peptides

| Antigen | Flourescent | Vendor |
|---|---|---|
| CD3 | APC/Cy7 | BioLegend |
| CD56 | BV510 | BioLegend |
| NKp46 | APC | BioLegend |

### Example 3

Expansion of iNK cells

iNK cells were seeded at 5×10⁴ cells/well in 96-well plate and expanded by anti-NKp46/CD2 antibody conjugated beads (Miltenyi) in Iscove's Modified Dulbecco's Media (IMDM) supplemented with 15% FBS, 4 mM L-glutamine, 100 U/ml penicillin, 100 μg/ml streptomycin, 50 μg/ml ascorbic acid 2-phosphate, 10 μg/ml human insulin, 5.5 μg/ml human transferrin, 6.7 ng/ml sodium selenite, 10 ng/ml IL-2, 10 ng/ml IL-7, 10 ng/ml IL-15 (Peprotech), 20 ng/ml IL-21 (Peprotech), 50 ng/ml IL-12 (Peprotech), 50 ng/ml IL-18 (MBL), 300 ng/ml anti-CD30 antibody (R&D systems), and 10 μM Z-VAD-FMK (Caspase inhibitor, R&D systems). After 4 days of culturing, cells were transferred to G-Rex24 at 1×10⁶ cells/well in IMDM medium supplemented with 15% FBS, 4 mM L-glutamine, 100 U/ml penicillin, 100 μg/ml streptomycin, 50 μg/ml ascorbic acid 2-phosphate, 10 μg/ml human insulin, 5.5 μg/ml human transferrin, 6.7 ng/ml sodium selenite, 10 ng/ml IL-2, 10 ng/ml IL-7, 10 ng/ml IL-15. The medium was changed every 2-3 days during additional 10 days of culturing. Repeated expandability of iNK cells was plotted as shown in FIG. 3. iNK cells which were differentiated from NKp46-untransduced iPSCs were expanded in the same method. As shown in Table 3, expansion rate of the iNK cells was low under this condition.

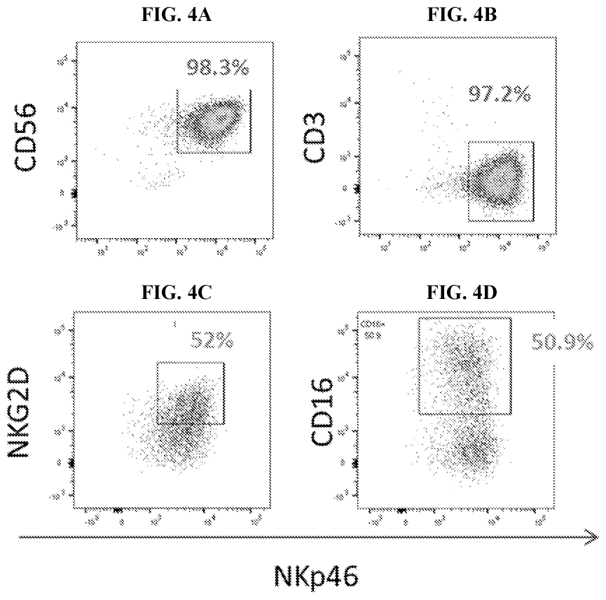

Expanded iNK cells were stained with antibodies as indicated in Table 4, and analyzed by flow cytometer for expression of NKG2D, CD56, CD16, and CD3 as shown in FIGS. 4A-4D. As shown in FIG. 4A, NKp46+ and CD56+ cells comprised about 98.3% of the cell population. As shown in FIG. 4B, NKp46+ and CD3− cells comprised about 97.2% of the cell population. As shown in FIG. 4C, NKp46+ and NKG2D+ cells comprised about 52% of the cell population. As shown in FIG. 4D, NKp46+ and CD16+ cells comprised about 50.9% of the cell population.

TABLE 3
Fold change (iNK cells differentiated
from NKp46-untransduced iPSCs)

| Days after first activation | Fold expansion |
|---|---|
| 0 | 1.0 |
| 3 | 0.6 |
| 7 | 0.8 |

TABLE 4
Stains used to detect peptides

| Antigen | Fluorescent | Vender |
|---|---|---|
| CD3 | APC/Cy7 | BioLegend |
| CD56 | BV510 | BioLegend |
| NKp46 | BV421 | BioLegend |
| NKG2D | APC | BioLegend |
| CD16 | PE/Cy7 | BioLegend |

### Example 4

### Effects of Anti-CD30 Antibody on iNK Cells Expansion

On Day 1, after two expansions by the method in Example 3, cryopreserved and harvested day-13 iNK cells were cultured for 1 day in Iscove's Modified Dulbecco's Media (IMDM) supplemented with 15% FBS, 4 mM L-glutamine, 100 U/ml penicillin, 100 μg/ml streptomycin, 50 μg/ml ascorbic acid 2-phosphate, 10 μg/ml human insulin, 5.5 μg/ml human transferrin, 6.7 ng/ml sodium selenite, 10 ng/ml IL-2, 10 ng/ml IL-7, 10 ng/ml IL-15 (Peprotech). The iNK cells were seeded at 2×10⁶ cells/well in T25 flask and expanded by plate-bound anti-NKp46 antibody (Monoclonal Mouse IgG2B Clone #195314, R&D systems) in IMDM supplemented with 15% FBS, 4 mM L-glutamine, 100 U/ml penicillin, 100 μg/ml streptomycin, 50 μg/ml ascorbic acid 2-phosphate, 10 μg/ml human insulin, 5.5 μg/ml human transferrin, 6.7 ng/ml sodium selenite, 10 ng/ml IL-2, 10 ng/ml IL-7, 10 ng/ml IL-15 (Peprotech), 20 ng/ml IL-21 (Peprotech), 50 ng/ml IL-12 (Peprotech), 50 ng/ml IL-18 (MBL), and 10 μM Z-VAD-FMK (Caspase inhibitor, R&D systems) in the presence or absence of 300 ng/ml anti-CD30 antibody (81316, R&D systems) on Day 0.

After 3 days of culturing, cells were transferred to G-Rex6 at 5×10⁶ cells/well in IMDM medium supplemented with 15% FBS, 4 mM L-glutamine, 100 U/ml penicillin, 100 μg/ml streptomycin, 50 μg/ml ascorbic acid 2-phosphate, 10 μg/ml human insulin, 5.5 μg/ml human transferrin, 6.7 ng/ml sodium selenite, 10 ng/ml IL-2, 10 ng/ml IL-7, and 10 ng/ml IL-15. The medium was changed every 2-3 days during additional 11 days of culturing and expandability was examined on Day 14 (3rd expansion). Repeated expandability was examined by the same protocol from Day 0 to Day 14 of Example 4 (4th expansion).

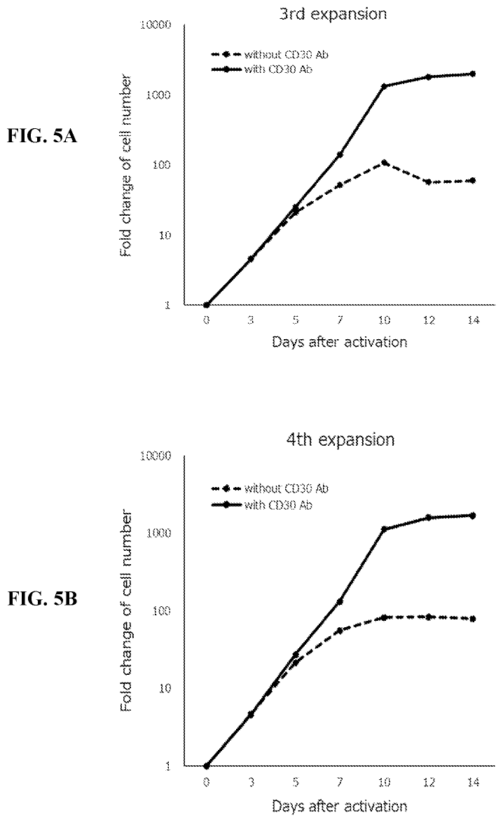

Expandability of iNK cells in the presence or absence of anti-CD30 antibody is shown in FIGS. 5A and 5B. As shown in Table 5, the expansion rate of iNK cells without anti-CD30 antibody was low compared to with anti-CD30 antibody condition.

TABLE 5
Fold change of 3^{rd} and 4^{th} expansion

| | |
|---|---|
| Without anti-CD30 Ab | With anti-CD30 Ab |

| Days after | 3rd | 4th | 3rd | 4th |
|---|---|---|---|---|
| activation | expansion | expansion | expansion | expansion |
| 0 | 1.0 | 1.0 | 1.0 | 1.0 |
| 3 | 4.5 | 4.7 | 4.6 | 4.6 |
| 5 | 21.0 | 21.4 | 25.0 | 27.2 |
| 7 | 51.7 | 55.8 | 139.0 | 131.7 |
| 10 | 107.5 | 82.0 | 1312.1 | 1120.8 |
| 12 | 56.7 | 83.1 | 1803.6 | 1582.4 |
| 14 | 59.8 | 78.8 | 1986.9 | 1690.8 |

### Example 5

### Effects of Anti-CD30 Antibody on In Vivo Persistence of iNK Cells

On Day 0 of the 4th expansion, harvested 3rd expansion Day 14 iNK cells were seeded at 1×10⁷ cells/well in T75 flask and activated by plate-bound anti-NKp46 antibody (R&D systems) in Iscove's Modified Dulbecco's Media (IMDM) supplemented with 15% FBS, 4 mM L-glutamine, 100 U/ml penicillin, 100 μg/ml streptomycin, 50 μg/ml ascorbic acid 2-phosphate, 10 μg/ml human insulin, 5.5 μg/ml human transferrin, 6.7 ng/ml sodium selenite, 10 ng/ml IL-2 (Peprotech), 10 ng/ml IL-7 (Peprotech), 10 ng/ml IL-15 (Peprotech), 20 ng/ml IL-21 (Peprotech), 50 ng/ml IL-12 (Peprotech), 50 ng/ml IL-18 (MBL), and 10 μM Z-VAD-FMK (Caspase inhibitor, R&D systems) in the presence or absence of 300 ng/ml anti-CD30 antibody (81316, R&D systems).

After 3 days of culturing, cells were transferred to G-Rex6 at 5×10⁶ cells/well in IMDM medium supplemented with 15% FBS, 4 mM L-glutamine, 100 U/ml penicillin, 100 μg/ml streptomycin, 50 μg/ml ascorbic acid 2-phosphate, 10 μg/ml human insulin, 5.5 μg/ml human transferrin, 6.7 ng/ml sodium selenite, 10 ng/ml IL-2, 10 ng/ml IL-7, 10 ng/ml IL-15 (Peprotech), 20 ng/ml IL-21 (Peprotech), 50 ng/ml IL-12 (Peprotech), and 50 ng/ml IL-18 (MBL) in the presence or absence of 300 ng/ml anti-CD30 antibody (81316, R&D systems).

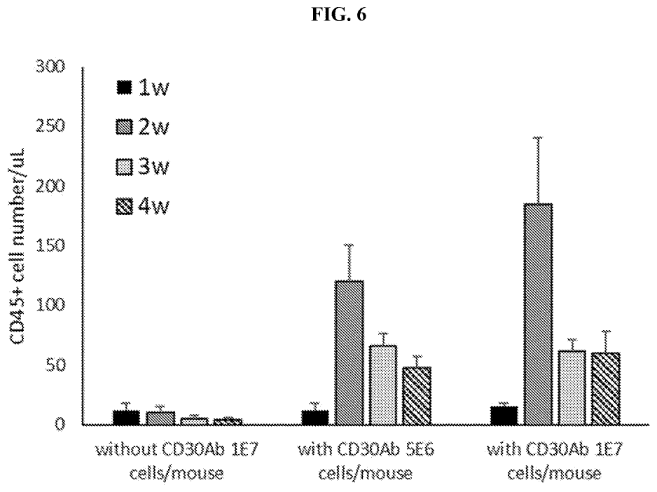

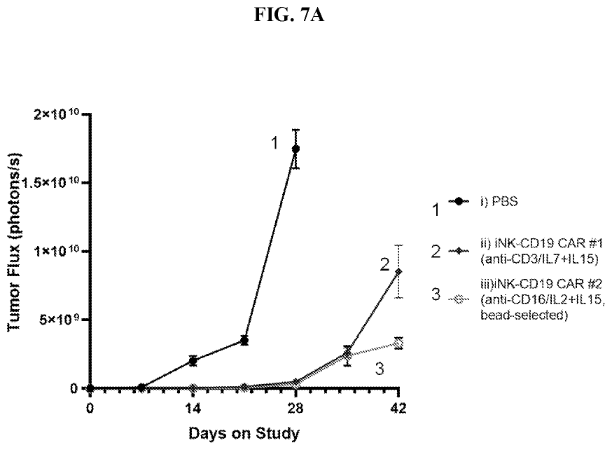

On Day 5, after activation, cells were transferred to G-Rex6 at 2×10⁷ cells/well and harvested on Day 7. These cells were cryopreserved in CS10 (BioLife Solutions) and thawed just before intravenous administration to 8-week-old NOG mice (0.5 or 1×10⁷ cells/mouse, N=3). Every week after administration, peripheral blood was collected and fixed by FACS lysing solution (BD) followed by staining with mouse CD45-PE antibody (BioLegend) and human CD45-BV510 antibody (BioLegend). The number of mouse CD45 negative and human CD45 positive cells was calculated as iNK cells persisted in vivo by flow cytometer. As shown in FIG. 6, iNK cells expanded with anti-CD30 antibody showed higher in vivo persistence.

### Example 6

Differentiation of iPS Cells into HP Cell Bulk

QHJI cells, an iPS cell strain, were derived from peripheral blood mononuclear cells of a healthy individual. On Day 0, QHJI cells dispersed in StemFit complete medium were seeded at 6×10⁵ cells/well in an ultra-low adhesion-treated 6-well plate under a low-oxygen (5% O₂) condition. The StemFit complete medium included 10 μM CHIR99021 and 50 PM Y-27632. On Day 1, the QHJI cells were dispersed in a hematopoietic progenitor cells (HPC) differentiation medium containing BMP4 (50 ng/ml), VEGF(50 ng/ml), bFGF(50 ng/ml), and ascorbic acid 2-phosphate (50 μg/mL). The HPC induction medium included StemPro34 supplemented with human insulin (10 μg/mL), human transferrin (5.5 μg/mL), sodium selenite (6.7 ng/ml), L-glutamine (2 mM) and a-monothioglycerol (0.4 mM).

On Day 2, SB431542 (at 6 μM in medium) was added to the culture medium (i.e., HPC differentiation medium containing cells), and the cells were cultured for 2 days. On Day 4, cells were re-dispersed in another medium containing VEGF(50 ng/ml), bFGF(50 ng/ml), SCF(50 ng/mL), and ascorbic acid 2-phosphate (50 μg/mL), and cultured for further 3 days. On Day 7, cells were exposed to another medium containing VEGF(50 ng/mL), bFGF(50 ng/mL), SCF(50 ng/mL), ascorbic acid 2-phosphate (50 μg/mL), TPO (30 ng/ml) and Flt3L (10 ng/mL), and cultured using this medium for additional 7 days. The medium was changed every 2-3 days during this 7 days-culture period.

### Example 7

Differentiation of HP Cell Bulk into a Population Comprising CD4− Cells

On Day 14, the cell population obtained from Example 6 (“HP cell bulk”), without undergoing any cell isolation, was seeded in 15 cm dishes at 3.12×10⁶ cells/dish, and cultured at 37° C. under 5% 02. It is noted that various seeding densities may be used, and the aforementioned seeding density is one example. Each 15 cm dish was coated with rh-DLL4/Fc chimera (Sino Biological) and RetroNectin (Takara Bio Inc). The medium was changed every 2-3 days during this culture period. MEMa (Thermo Fisher Scientific (Gibco)) supplemented with 15% FBS, 4 mM L-glutamine, 100 U/mL penicillin, 100 μg/ml streptomycin, 55 μM 2-mercaptoethanol, 50 μg/mL ascorbic acid 2-phosphate, 10 μg/mL human insulin, 5.5 μg/mL human transferrin, 6.7 ng/mL sodium selenite, 50 ng/mL SCF, 50 ng/ml IL-7, 50 ng/mL Flt3L, 100 ng/ml TPO, 15 μM SB203580, 30 nM SDF-la was used as the medium to culture these cells. On Day 21, the cells were passaged to new 15 cm dishes freshly coated with hDLL4/RetroNectin. On Day 28, the cells were further passaged to new 15 cm dishes freshly coated with hDLL4/RetroNectin. On Day 35, all cells including CD4− cells, among others, were harvested.

### Example 8

### Transduction of the NK Cells

### A. Differentiation of Bulk to NK Cell Bulk

On Day 35, a cell population comprising CD4− cells, i.e., cells obtained from Example 7 without undergoing any cell isolation, was seeded in a 48-well plate at 1×10⁶ cells/well, and cultured under 5% CO₂ at 37° C. for three days. MEMa medium supplemented with 15% FBS, 4 mM L-glutamine, 100 U/mL penicillin, 100 ng/mL streptomycin, 50 μg/mL ascorbic acid 2-phosphate, 10 μg/mL human insulin, 5.5 μg/mL human transferrin, 6.7 ng/mL sodium selenite, 500 ng/ml anti-CD3 antibody (UCHT1, R&D Systems), 10 ng/ml IL-2, and 10 ng/ml IL-7 was used as a culture medium.

On Day 38, the cells were dispersed in another MEMa medium supplemented with 15% FBS, 4 mM L-glutamine, 100 U/mL penicillin, 100 ng/ml streptomycin, 50 μg/mL ascorbic acid 2-phosphate, 10 μg/mL human insulin, 5.5 μg/mL human transferrin, 6.7 ng/ml sodium selenite, 10 ng/mL IL-2, and 10 ng/mL IL-7 was used as a culture medium. On Day 42, all cells including NK cells (“NK cell bulk”) were harvested.

### B. Activation and Expansion of the NK Cells

On Day 41, a tissue culture vessel was coated with retronectin (0.15 μg/cm²) and either anti-CD3 antibody (UCHT1, R&D Systems) (0.6 μg/cm²) or anti-CD16 antibody (3G8, Biogems) (0.6 μg/cm²) for 16 h at 4° C. On Day 42, the harvested NK cells from Example 8A were resuspended to 1×10⁵ cells/mL in IMDM medium (Thermo Fisher Scientific (Gibco)), supplemented with 15% FBS, 4 mM L-glutamine, 100 U/mL penicillin, 100 ng/ml streptomycin, 50 μg/mL ascorbic acid 2-phosphate, 10 μg/mL human insulin, 5.5 μg/mL human transferrin, 6.7 ng/mL sodium selenite, 500 ng/ml anti-CD3 antibody (UCHT1), 10 ng/ml IL-7, 50 ng/ml IL-15, 50 ng/mL IL-18, 20 ng/ML IL-21, 10 μM Z-VAD-FMK caspase inhibitor, and 300 ng/mL anti-CD30 antibody (81316, R&D Systems) and transferred to the anti-CD3 coated vessels to be cultured for three days.

On Day 45, the cells were collected and either transferred to a gammaretrovirus-coated vessel for viral transduction, described below in Example 8C, or dispersed in new IMDM medium supplemented with 15% FBS, 4 mM L-glutamine, 100 U/mL penicillin, 100 ng/ml streptomycin, 50 μg/mL ascorbic acid 2-phosphate, 10 μg/mL human insulin, 5.5 μg/mL human transferrin, 6.7 ng/ml sodium selenite, 10 ng/mL IL-7 or 10 ng/ml IL-2, and 10 ng/ml IL-15, and transferred to a G-Rex vessel (Wilson Wolf) to be cultured an additional 11 days. IL-7 was used with anti-CD3 antibody, and IL-2 was used with anti-CD16 antibody. 50-80% fresh medium changes were performed every 2-3 days with the same medium composition, and cells were split to additional G-Rex vessels if the cell concentration exceeded 4×10⁷ cells/cm². On Day 56, the cells were harvested for final use.

If necessary to obtain a sufficient number of cells, the 15-day process described here (Day 41-Day 56) was repeated multiple times to increase the total number of NK cells generated. For the iNK-CD19 CAR cells, the cells went through 5 activation rounds (5×15 day process), and the CAR transduction was performed in the 2nd activation. For the INK-Meso CAR cells, the cells went through 4 activation rounds (4×15 day process), and the CAR transduction was performed in the 3rd activation.

### C. Gammaretrovirus Transduction of the NK Cells.

For gammaretrovirus transduction of the NK cells, on Day 44, tissue culture vessels were coated with retronectin (10.5 μg/cm²) for 16 h at 4° C. On Day 45, gammaretrovirus was incubated on the retronectin-coated vessels for 2 h at 32° C., and subsequently harvested. On Day 45, NK cells described in Example 8B were centrifuged on to the retronectin/gammaretrovirus-coated vessels at 300×g for 5 minutes at 32° C. and cultured overnight. If necessary to achieve a higher percentage of transduced cells, the steps described from Day 44-Day 45 were repeated to perform the gammaretrovirus transduction twice. On Day 46 (or Day 47 if the gammaretrovirus transduction was performed twice), the cells were collected, dispersed in IMDM medium supplemented with 15% FBS, 4 mM L-glutamine, 100 U/mL penicillin, 100 ng/ml streptomycin, 50 μg/mL ascorbic acid 2-phosphate, 10 μg/mL human insulin, 5.5 μg/mL human transferrin, 6.7 ng/mL sodium selenite, 10 ng/ml IL-7 or 10 ng/ml IL-2, and 10 ng/ml IL-15, and transferred to a G-Rex vessel (Wilson Wolf). Subsequently, 75% medium changes were performed every 2-3 days with the same medium composition and cells were split to additional G-Rex vessels if the cell concentration exceeded 4×10⁷ cells/cm². On Day 56, the cells were harvested for final use.

If necessary, the 15-day process described Example 8B (Day 41-Day 56) was additionally repeated multiple times to increase the total number of iNK cells generated. For the iNK-CD19 CAR cells, the cells went through 5 activation rounds (5×15-day process-the same process as Day 41-Day 56), and the CAR and IL-15Ra/IL-15 genes transduction was performed in the 2nd activation. For the iNK-Meso CAR cells, the cells went through 4 activation rounds (4×15—day process—the same process as Day 41-Day 56), and the CAR and IL-15Ra/IL-15 genes transduction was performed in the 3rd activation.

Table 6 shows different culture conditions and the CAR+ and total cell counts based on each culture condition. Those cells are used for the in vivo study in Example 10.

TABLE 6
Culture Conditions.

| # | Activation/Expansion Groups | CAR/UTD | Cell (×10\hat{ }6/mouse) |
|---|---|---|---|
| 1 | PBS | | — |
| 2 | aCD3/IL7 + IL15 | CD19-mbIL15 | 14.99 |
| 3 | aCD16/IL2 + IL15 | CD19-mbIL15 | 12.38 |

### Example 9

### CAR-NK Cell Preparation

NK cells were further modified to express one or more CARs. NK cells were modified by: (1) synthesizing an anti-CD19 CAR gene or anti-Mesothelin CAR gene and an IL-15Ra/IL-15 gene; (2) preparing a retrovirus vector containing anti-CD 19 CAR gene or anti-Mesothelin CAR gene and IL-15Ra/IL-15 genes; and (3) transducing the NK cells with the retrovirus vector containing anti-CD 19 CAR gene or anti-Mesothelin CAR gene and IL-15Ra/IL-15 genes.

### CAR/IL15-NK Cell Preparation

An anti-CD19 CAR gene was prepared by synthesizing oligopeptides that are designed to be arranged from N-terminal as shown in Table 6.

TABLE 7
Oligopeptides.

| Sequence from | | Number of |
|---|---|---|
| N-terminal | Genes | Amino acids |
| 1 | Lead sequence of the heavy chain | 22 |
| | of an immunoglobulin | |
| 2 | Variable domain in the light chain | 104 |
| | of the anti-CD19 | |
| | antibody(FMC60) | |
| 3 | GGGGS linker | 15 |
| 4 | Variable domain in the heavy | 120 |
| | chain of the anti-CD19 | |
| | antibody(FMC60) | |
| 5 | CD8 derived sequence | 83 |
| | (Containing transmembrane | |
| | domain) | |
| 6 | Intracellular domain of 4-1BB | 47 |
| 7 | Intracellular domain of CD3 zeta | 112 |

An anti-CD 19 CAR was constructed in accordance with US2021/0292713, which is incorporated herein by reference in its entirety. An anti-Mesothelin CAR was constructed in accordance with WO2023/009700, which is incorporated herein by reference in its entirety.

### Example 10

### In Vivo Anti-Tumor Activity of INK-CD19 CAR

Luciferase expressing Nalm6 cells (ATCC; cancer cells) (5×10⁵ cells) were transplanted into NOD/Shi-scid, IL-2R gamma null mice (“NSG mice”) via the tail vein. The 8-9 week old female NSG mice were sourced from The Jackson Laboratory. On Day 4, four days after the transplantation of Nalm6 cells, the iNK-CD19 CAR cells (10×10⁶ CAR+ cells) dispersed in 0.2 ml PBS or just 0.2 ml of PBS without cells were administered to the Nalm6-transplanted NSG mice via the tail vein. After the administration of iNK-CD19 CAR cells or PBS, luciferin was administered to the mice via the tail vein. Luciferase activity was measured using IVIS imaging system (PerkinElmer) for 42 days.

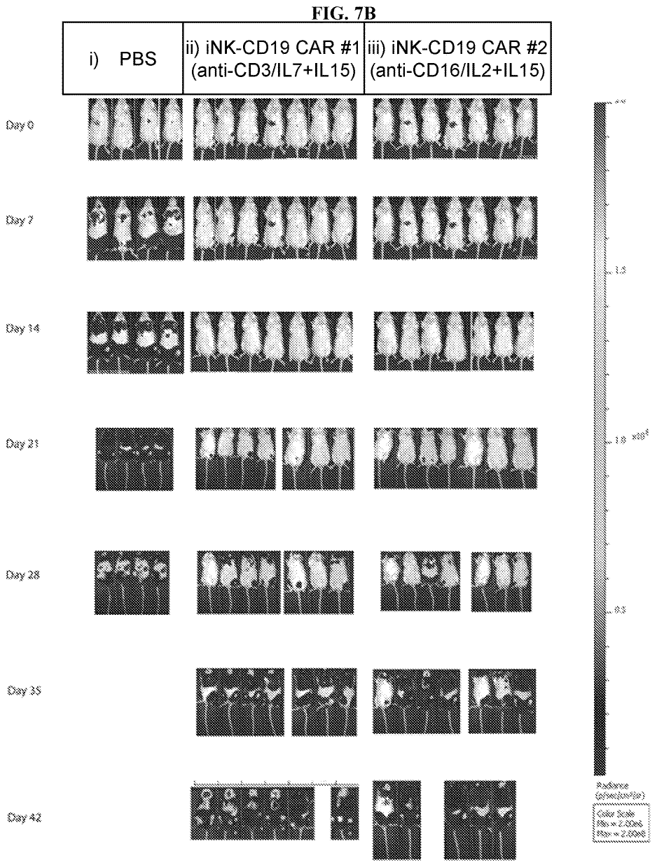

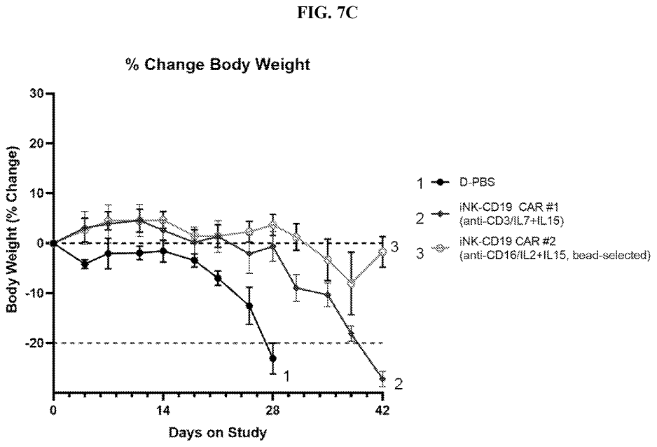

FIGS. 7A and 7B shows the anti-tumor efficacy of (1) PBS treated (vehicle control), (2) iNK-CD19 CAR cells generated with the cell cultures described in Examples 6-8 and anti-CD3 antibody activation (“iNK-CD19 CAR #1), and (3) iNK-CD19 CAR cells generated with the cell culture methods described in Examples 6-8 but excluding Example 8A and with anti-CD-16 antibody activation (“iNK-CD19 CAR #2). While detectable tumor growth was observed in the PBS vehicle treated group by Day 14 post-tumor cell infusion and progressively increased from that time point, in both iNK-CD19 CAR groups, significant expansion of the tumor cells was not observed until Day 28. FIG. 7C depicts the percent change in body weight of the mice over the course of the study. While the PBS control group exhibited significant (>20%) body weight loss by Day 28, the iNK-CD19 CAR #1 group did not achieve this endpoint until Day 42, and the INK-CD19 CAR #2 group never reached this level of body weight loss. Together, these data demonstrate the capacity of the iNK-CD19 CAR cells to reduce the proliferation of Nalm6 tumor cells in vivo.

### Example 11

In Vitro Repeat Killing Ability of iNK-Mesothelin CAR Cells

A repeat antigen stimulation (RAS) assay was performed using red fluorescent protein (RFP)-expressing GSU tumor cells that express high levels of Mesothelin protein, and iNK-Mesothelin CAR cells (INK-Meso CAR cells). GSU-RFP cells were seeded at 1×10⁵ cells per well in a 96 well plate and allowed to grow overnight. The following day, (1) no cells, (2) 1×10⁵ untransduced (UTD) iNK, or (3) 1×10⁵ iNK-Meso CAR cells were added to each well. The RFP-positive surface area of each well was measured every 4 hours by Incucyte.

To repeatedly challenge the iNK cells, an additional 1×10⁵ cells GSU-GFP cells were added to each well every 2-3 days. This process is referred to herein as a “challenge.” Four challenges were performed in total.

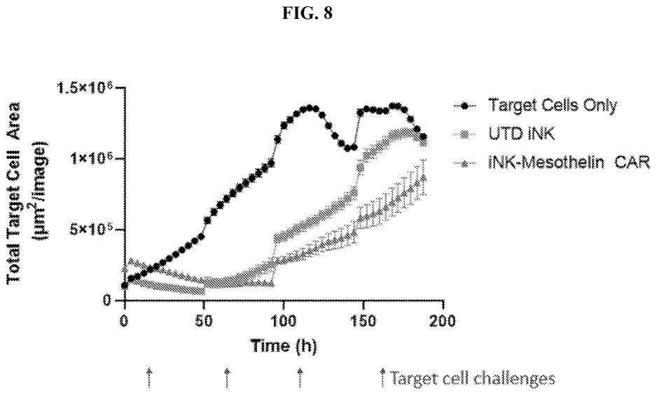

FIG. 8 depicts the growth of the GSU tumor cells, measured via total RFP-positive surface area per image per well, over the course of the study. The GSU cells continuously grew in the tumor-cell-only condition until ~120 hours, when the wells became overconfluent. In comparison, the UTD and iNK-Meso CAR cells significantly decreased the rate of tumor cell growth. The UTD iNK cells completely inhibited tumor growth for one challenge, while the iNK-Meso CAR cells controlled tumor cell growth for two challenges. These results demonstrate that while the UTD iNK cells have some innate tumor control capacity, CAR transduction further increases the ability of the iNK cells to kill tumor cells.

### Example 12

### In Vivo Anti-Tumor Activity of INK-Meso CAR

Luciferase expressing GSU cells (1×10⁶ cells) were transplanted into NOD/Shi-scid, IL-2R gamma null mice (“NSG mice”) via intraperitoneal injection. The female, 8-9 week old NSG mice were sourced from The Jackson Laboratory.

Four days after the transplantation of Nalm6 cells, the iNK-Meso CAR cells (20 ×10⁶ cells) dispersed in 0.2 ml PBS or 0.2 ml of PBS with no cells were administered to the GSU-transplanted NSG mice via the tail vein. After the administration of iNK-Meso cells or PBS, luciferin was administered to the mice via intraperitoneal injection. Luciferase activity was measured using the IVIS imaging system (PerkinElmer) for 31 days. Table 8 below shows the two study groups: PBS alone, and iNK-Meso CAR cells.

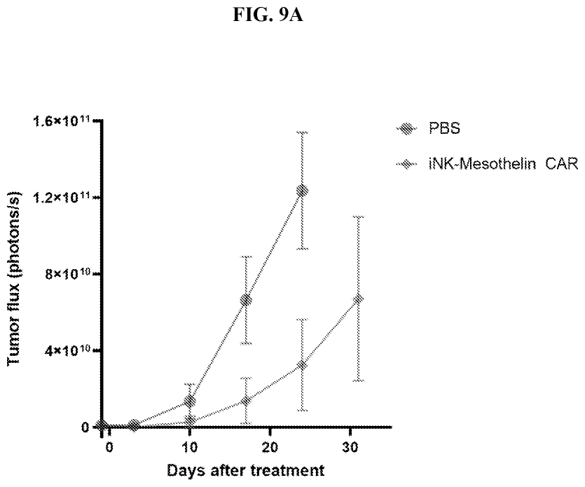

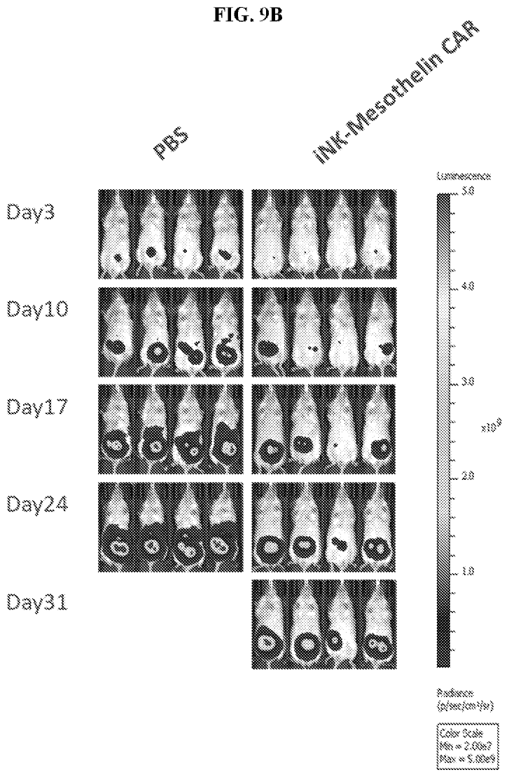

FIGS. 9A and 9B show the tumor growth measured by luciferase activity in mice treated with (1) PBS (vehicle control) or (2) iNK-Meso CAR cells. Treatment with the iNK-Meso CAR cells significantly delayed the tumor growth relative to the PBS control, indicating that the iNK-Meso CAR cells have anti-tumor activity.

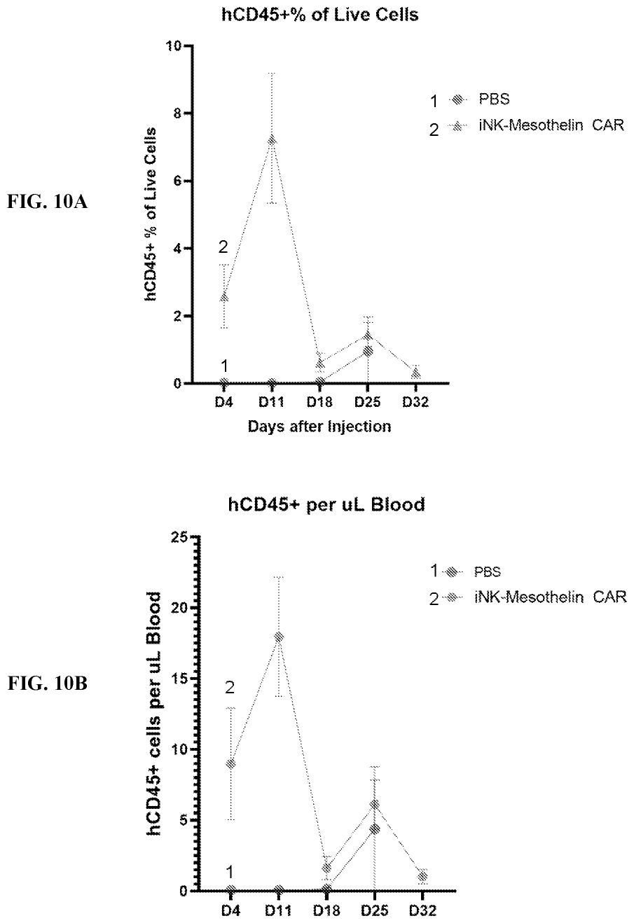

In addition, peripheral blood samples were collected to assess in vivo iNK-Meso CAR cell proliferation by flow cytometry for human CD45, which is only present on the transfused iNK-Meso CAR cells. FIGS. 10A and 10B show the cellular growth kinetics of the iNK-Meso CAR cells over time, indicated by the percentage of the total live cells that are human CD45⁺ cells, and by the absolute number of human CD45⁺ cells per μL of mouse peripheral blood. Some iNK-Meso CAR cells were detectable at Day 4, the earliest time point assessed. However iNK-Meso CAR cells levels continued to increase, ultimately peaking at Day 11. The increase in detectable iNK cells from Day 4 to Day 11 indicates that the iNK-Meso CAR cells can proliferate in an in vivo mouse model system.

TABLE 8

| Group # | CAR | Total # cells per mouse |
|---|---|---|
| 1 | PBS | — |
| 2 | Meso CAR* | 2.03E+07 |

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed:

1. A method of expanding a CD56+/CD3− cell comprising:
culturing the CD56+/CD3− cell in the presence of an NKp46 agonist, wherein the CD56+/CD3−cell comprises at least one exogenous nucleic acid molecule encoding one or more NKp46 polypeptides.

2. The method of claim 1, wherein the CD56+/CD3− cell is derived from a pluripotent stem cell.

3. The method of claim 2, further comprising transducing the pluripotent stem cell with at least one exogenous nucleic acid molecule encoding one or more NKp46 polypeptides.

4. The method of claim 2 or 3, wherein the pluripotent stem cell is an induced pluripotent stem cell (iPSC).

5. The method of any one of claims 1 to 4, wherein the NKp46 agonist is an anti-NKp46 antibody.

6. The method of any one of claims 1 to 5, wherein the CD56+/CD3− cell is cultured in the presence of the NKp46 agonist and at least one cytokine.

7. The method of claim 6, wherein the at least one cytokine comprises interleukin-(IL) 2, IL-7, IL-15, IL-12, IL-18, IL-21, or any combination thereof.

8. The method of any one of claims 1 to 7, wherein the CD56+/CD3− cell further comprises at least one exogenous nucleic acid molecule encoding (i) one or more chimeric antigen receptors (CARs), and/or (ii) one or more cytokines or mimetics thereof.

9. The method of claim 8, wherein the cytokine or the mimetic thereof is an IL-15.

10. The method of claim 9, wherein the IL-15 is linked to IL-15Ra to form a fusion protein.

11. A method of producing a modified CD56+/CD3− cell comprising:
(A) providing an ex vivo bulk cell production comprising hematopoietic progenitor cells (HPCs), comprising at least one exogenous nucleic acid molecule encoding one or more NKp46 polypeptides, and
(B) differentiating the HPCs to generate one or more CD56+/CD3− cells.

12. The method of claim 11, further comprising:
(C) culturing the CD56+/CD3− cells in the presence of an NKp46 agonist.

13. The method of claim 11 or 12, wherein the HPCs are derived from one or more pluripotent stem cells.

14. The method of claim 13, further comprising transducing the one or more pluripotent stem cells with at least one exogenous nucleic acid molecule encoding one or more NKp46 polypeptides.

15. The method of claim 13 or 14, wherein the pluripotent stem cells are iPSCs.

16. The method of any one of claims 11 to 15, wherein the NKp46 agonist is an anti-NKp46 antibody.

17. The method of any one of claims 11 to 16, wherein the CD56+/CD3− cell is cultured in presence of the NKp46 agonist and at least one cytokine.

18. The method of claim 17, wherein the at least one cytokine comprises IL-2, IL-7, IL-15, IL-12, IL-18, IL-21, or any combination thereof.

19. The method of any one of claims 11 to 18, wherein the CD56+/CD3− cells further comprise at least one exogenous nucleic acid molecule encoding (i) one or more chimeric antigen receptors (CARs), and/or (ii) one or more cytokines or mimetics thereof.

20. The method of claim 19, wherein the cytokine or the mimetic thereof is an IL-15.

21. The method of claim 20, wherein the IL-15 is linked to IL-15Ra to form a fusion protein.

22. The method of any one of claims 1 to 21, further comprising culturing the CD56+/CD3− cell in the presence of a CD30 agonist.

23. The method of claim 22, wherein the CD30 agonist is an anti-CD30 antibody.

24. A CD56+/CD3− cell which is obtained through the method of any one of claims 1 to 23.

25. A pharmaceutical composition comprising the CD56+/CD3− cell of claim 24 and an excipient.

26. A method of treating a disease or condition in a subject in need thereof, comprising administering to the subject the CD56+/CD3− cell of claim 24 or the pharmaceutical composition of claim 25.

27. A method of expanding a CD56+/CD3− cell comprising culturing the CD56+/CD3− cell in the presence of a CD30 agonist.

28. The method of claim 27, wherein the CD56+/CD3− cell is derived from a pluripotent stem cell.

29. The method of claim 28, wherein the pluripotent stem cell is an induced pluripotent stem cell (iPSC).

30. The method of any one of claims 27 to 29, wherein the CD30 agonist is an anti-CD30 antibody.

31. The method of any one of claims 27 to 30, wherein the CD56+/CD3− cell is cultured in the presence of the CD30 agonist and at least one cytokine.

32. The method of claim 31, wherein the at least one cytokine comprises IL-2, IL-7, IL-15, IL-12, IL-18, IL-21, or any combination thereof.

33. A CD56+/CD3− cell which is obtained through the method of any one of claims 27 to 32.

34. A pharmaceutical composition comprising the CD56+/CD3− cell of claim 33 and an excipient.

35. A method of treating a disease or condition in a subject in need thereof, comprising administering to the subject the CD56+/CD3− cell of claim 33 or the pharmaceutical composition of claim 34.
